(19) United States
(12) Patent Application Publication
(10) Pub. No.: US 2025/0281298 A1
(43) Pub. Date: Sep. 11, 2025

(54) PAPILLARY MUSCLE APPROXIMATION

(71) Applicant: EDWARDS LIFESCIENCES CORPORATION, Irvine, CA (US)

(72) Inventors: Felino V. Cortez, JR., Bowie, MD (US); Nancy Ling Chung, Edgewater, MD (US); Stephen Cournane, Severn, MD (US); Stephen Epstein, Millersville, MD (US); Jyoti B. Rao, Brea, CA (US)

(21) Appl. No.: 19/220,523

(22) Filed: May 28, 2025

Related U.S. Application Data
(63) Continuation of application No. PCT/US23/79574, filed on Nov. 14, 2023.
(60) Provisional application No. 63/385,187, filed on Nov. 28, 2022.

(51) Int. Cl. A61F 2/24

(52) U.S. Cl. CPC A61F 2/2487

(57) ABSTRACT

A surgical device includes a handle, a shaft extending distally from the handle, and a curved arm projecting from a distal end of the shaft. The surgical device can be used for papillary muscle approximation procedures.

### CROSS REFERENCE TO RELATED APPLICATIONS

This application is a continuation of International Patent Application No. PCT/US23/79574, filed Nov. 14, 2023, which claims the benefit of U.S. Patent Application No. 63/385,187, filed Nov. 28, 2022, the entire disclosures all of which are incorporated by reference for all purposes.

### BACKGROUND

The present disclosure generally relates to the field of valve correction. Heart valve dysfunction can result in regurgitation and other complications due to valve prolapse from failure of valve leaflets to properly coapt. For atrioventricular valves, papillary muscle position can affect the ability of valve leaflets to function properly.

### SUMMARY

Described herein are devices, methods, and systems that facilitate the manipulation of papillary muscles to improve valve leaflet coaptation. Devices associated with the various examples of the present disclosure can include instruments having curved distal papillary-muscle-circumscribing arms configured to facilitate the wrapping of papillary muscles with muscle-approximating bands. Devices associated with the various examples of the present disclosure can further include spacer instruments and devices that can be used to establish/define a distance between papillary muscles in connection with certain papillary muscle approximation procedures.

For purposes of summarizing the disclosure, certain aspects, advantages and novel features have been described. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 provides a cut-away view of a human heart.

FIG. 2A provides a cut-away view of a ventricle and atrium of an example heart.

FIG. 2B provides an overhead view of a heart valve of a heart in a healthy condition.

FIG. 3A provides a cut-away view of a heart experiencing mitral regurgitation.

FIG. 3B provides an overhead view of a heart valve in a state in which mitral regurgitation is present.

FIG. 4 shows a cutaway view of a ventricle including papillary muscles that have been approximated using a papillary-muscle-approximation procedure in accordance with some examples.

FIGS. 5A-5G provide views of an anatomy-circumscribing instrument in accordance with some examples.

FIGS. 6-1, 6-2, 6-3, 6-4, and 6-5 illustrate a flow diagram for a process for capturing papillary muscle anatomy using a circumscriber instrument in accordance with some examples.

FIGS. 7-1, 7-2, 7-3, 7-4, 7-5, 7-6, and 7-7 provide images of the circumscriber instrument and certain anatomy corresponding to operations of the process of FIGS. 6-1, 6-2, 6-3, 6-4, and 6-5 in accordance with some examples.

FIGS. 8A and 8B show perspective and side views, respectively, of an anatomy-circumscribing instrument in accordance with some examples.

FIGS. 9-1 and 9-2 show anatomy-circumscribing instruments having distal articulating features in accordance with some examples.

FIG. 10 shows a papillary-muscle-approximation kit in accordance with some examples.

FIG. 11 shows a cutaway view of a heart ventricle having a spacer device placed between papillary muscles thereof in accordance with some examples.

FIG. 12 shows spacer instrumentation in accordance with some examples.

FIGS. 13-1 and 13-2 illustrate a flow diagram for a process for using a spacer for papillary muscle approximation in accordance with some examples.

FIGS. 14-1, 14-2, 14-3, and 14-4 provide images of spacer instrumentation and certain anatomy corresponding to operations of the process of FIGS. 13-1 and 13-2 in accordance with some examples.

FIG. 15 shows a balloon spacer instrument in accordance with some examples.

FIG. 16 shows a graph demonstrating distance/diameter of a compliant balloon spacer device relative to balloon inflation pressure in accordance with some examples.

FIG. 17 shows a caliper-type papillary muscle spacer instrument in accordance with some examples.

### DETAILED DESCRIPTION

The headings provided herein are for convenience only and do not necessarily affect the scope or meaning of the claimed invention.

Although certain preferred examples are disclosed below, it should be understood that the inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain reference numbers are re-used across different figures of the figure set of the present disclosure as a matter of convenience for devices, components, systems, features, and/or modules having features that may be similar in one or more respects. However, with respect to any of the examples disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to only the numeric portion (e.g., ‘10’) may refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present written description to a feature ‘10’ may be understood to refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Heart Valve Disease

Functional mitral valve regurgitation (FMR) is a disease that occurs when the left ventricle of the heart is distorted or dilated, displacing the papillary muscles that support the leaflets/cusps of the mitral valve. When the valve leaflets can no longer come together to close the annulus, blood may flow back into the atrium.

The anatomy of the heart is described below to assist in the understanding of certain inventive concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles may be prompted by signals generated by the electrical system of the heart.

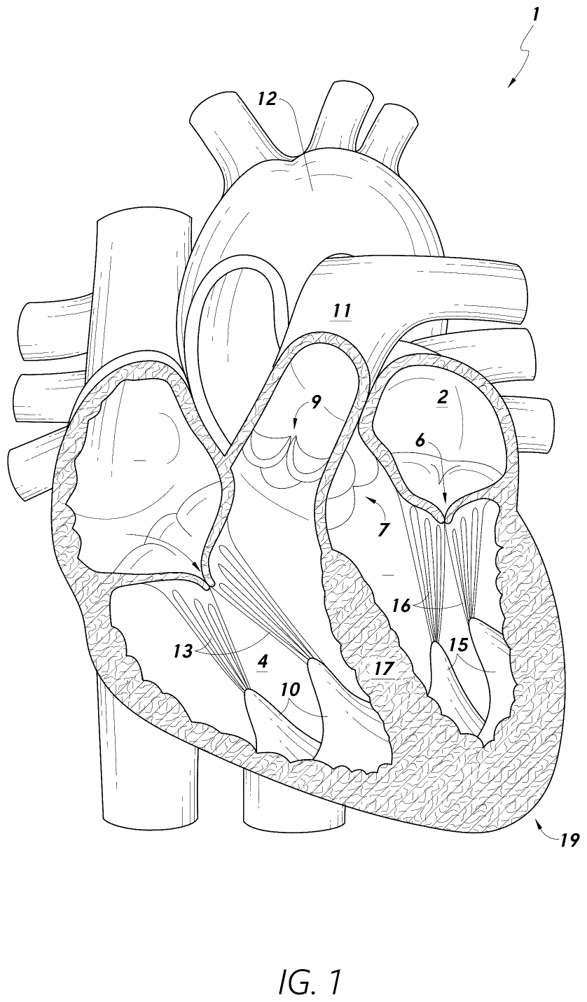

FIG. 1 illustrates an example representation of a heart 1 having various features relevant to certain embodiments of the present inventive disclosure. The heart 1 includes four chambers, namely the left atrium 2, the left ventricle 3, the right ventricle 4, and the right atrium 5. A wall of muscle 17, referred to as the septum, separates the left 2 and right 5 atria and the left 3 and right 4 ventricles. The inferior tip 19 of the heart 1 is referred to as the apex (or apex region) and is generally located on the midclavicular line, in the fifth intercostal space.

The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (e.g., systole) and open during ventricular expansion (e.g., diastole). The valves of the heart 1 further include the pulmonary valve 9, which separates the right ventricle 4 from the pulmonary artery 11. The pulmonary valve 9 is generally configured to open during systole so that blood may be pumped toward the lungs, and close during diastole to prevent blood from leaking back into the heart from the pulmonary artery. The pulmonary valve 9 generally has three cusps/leaflets, wherein each one may have a crescent-type shape.

The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

Heart valves may generally comprise a relatively dense fibrous ring, referred to herein as the annulus, as well as a plurality of leaflets or cusps attached to the annulus. Generally, the size of the leaflets or cusps may be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps ideally are brought into apposition to each other, thereby closing the flow passage.

The atrioventricular (e.g., mitral 6 and tricuspid 8) heart valves may further comprise a collection of chordae tendineae 13, 16 and papillary muscles 10, 15 for securing the leaflets of the respective valves to promote and/or facilitate proper coaptation of the valve leaflets and prevent prolapse thereof. The papillary muscles, for example, may generally comprise finger-like projections from the ventricle wall. With respect to the tricuspid valve 8, the normal tricuspid valve may comprise three leaflets and three corresponding papillary muscles 10 (two shown in FIG. 1 for clarity). The leaflets of the tricuspid valve may be referred to as the anterior, posterior and septal leaflets, respectively. The tricuspid valve leaflets are connected to the papillary muscles 10 by the chordae tendineae 13, which are disposed in the right ventricle 4 along with the papillary muscles 10. Although tricuspid valves are described herein as comprising three leaflets, it should be understood that tricuspid valves may occur with two or four leaflets in certain patients and/or conditions; the principles relating to papillary muscle repositioning disclosed herein are applicable to atrioventricular valves having any number of leaflets and/or papillary muscles associated therewith.

The right ventricular papillary muscles 10 originate in the right ventricle wall and attach to the anterior, posterior, and septal leaflets of the tricuspid valve 8, respectively, via the chordae tendineae 13. The papillary muscles 10 of the right ventricle 4 may have variable anatomy; the anterior papillary may generally be the most prominent of the papillary muscles. The papillary muscles 10 may serve to secure the leaflets of the tricuspid valve 8 to prevent prolapsing of the leaflets into the right atrium 5 during ventricular systole. Tricuspid regurgitation can be the result of papillary dysfunction or chordae rupture.

With respect to the mitral valve 6, a normal mitral valve may comprise two leaflets (anterior and posterior) and two corresponding papillary muscles 15. The papillary muscles 15 originate in the left ventricle wall and project into the left ventricle 3. Generally, the anterior leaflet may cover approximately two-thirds of the valve annulus. Although the anterior leaflet covers a greater portion of the annulus, the posterior leaflet may comprise a larger surface area in certain anatomies.

The valve leaflets of the mitral valve 6 may be prevented from prolapsing into the left atrium 2 by the action of the chordae tendineae 16 tendons connecting the valve leaflets to the papillary muscles 15. The relatively inelastic chordae tendineae 16 are attached at one end to the papillary muscles 15 and at the other to the valve leaflets; chordae tendineae from each of the papillary muscles 15 are attached to a respective leaflet of the mitral valve 6. Thus, when the left ventricle 3 contracts, the intraventricular pressure forces the valve to close, while the chordae tendineae 16 keep the leaflets coapting together and prevent the valve from opening in the wrong direction, thereby preventing blood from flowing back to the left atrium 2. The various chords of the chordae tendineae may have different thicknesses, wherein relatively thinner chords are attached to the free leaflet margin, while relatively thicker chords (e.g., strut chords) are attached farther away from the free margin. The bases of the papillary muscles 15 can be joined to the ventricle wall by trabeculae carneae tissue, which may be referred to herein simply as ‘trabeculation,’ or ‘trabeculae’ and generally comprises irregular muscular columns that project from the inner surface of the right and left ventricles of the heart.

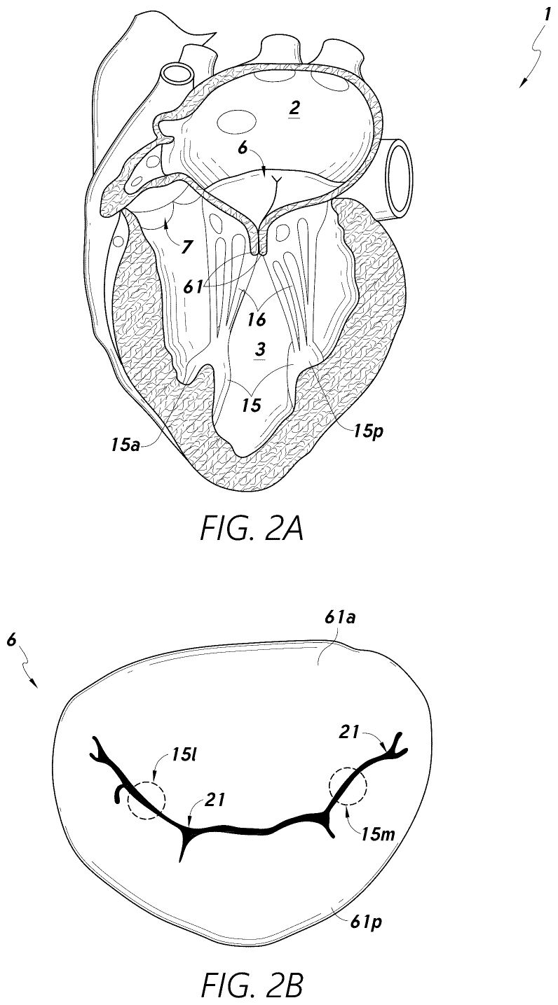

FIG. 2A provides a cross-sectional view of the left ventricle 3 and left atrium 2 of an example heart 1. While some example devices and/or methods are described herein with respect to the left ventricle 3, mitral valve 6, and/or left atrium 2, such devices and/or methods may be applied to and/or performed within other areas of the heart, including the right ventricle 4, right atrium 5, and/or tricuspid valve 8. The diagram of FIG. 2A shows the mitral valve 6. In FIG. 2A, the disposition of the valve 6, papillary muscles 15 and/or chordae tendineae 16 may be illustrative as providing for proper coapting of the valve leaflets to advantageously at least partially prevent regurgitation and/or undesirable flow into the left atrium from the left ventricle 3 and vice versa. Although a mitral valve 6 is shown in FIG. 2A and various other figures provided herewith and described herein in the context of certain examples of the present disclosure, it should be understood that papillary muscle repositioning principles disclosed herein may be applicable with respect to any atrioventricular valve and associated anatomy (e.g., papillary muscles, chordae tendineae, ventricle wall, etc.), such as the tricuspid valve.

As described above, with respect to a healthy heart valve as shown in FIG. 2A, the valve leaflets 61 may extend inward from the valve annulus and come together in the flow orifice to permit flow in the outflow direction (e.g., the downward direction in FIG. 2A) and prevent backflow or regurgitation toward the inflow direction (e.g., the upward direction in FIG. 2A). For example, during atrial systole, blood flows from the atria 2 to the ventricle 3 down the pressure gradient, resulting in the chordae tendineae 16 being relaxed due to the atrioventricular valve 6 being forced open. When the ventricle 3 contracts during ventricular systole, the increased blood pressures in both chambers may push the valve 6 closed, preventing backflow of blood into the atria 2. Due to the lower blood pressure in the atria compared to the ventricles, the valve leaflets may tend to be drawn toward the atria. The chordae tendineae 16 can serve to tether the leaflets and hold them in a closed position when they become tense during ventricular systole. The papillary muscles 15 provide structures in the ventricles for securing the chordae tendineae 16 and therefore allowing the chordae tendineae 16 to hold the leaflets in a closed position.

FIG. 2B shows an en face, or overhead, view of the mitral valve 6, with example positions of the heads of the papillary muscles 15 within the left ventricle 3 shown in dashed-line for reference. The papillary muscles 15 may include a first papillary muscle 15l (e.g., an ‘anterolateral’ papillary muscle, or ‘lateral’ papillary muscle, which may be primarily tethered to the anterior leaflet 61a, for example) and a second papillary muscle 15m (e.g., the ‘posteromedial’ papillary muscle, or ‘medial’ papillary muscle, which may be primarily tethered to the posterior leaflet 61p, for example). Each of the lateral papillary muscle 15l and medial papillary muscle 15m may provide chordae tendineae 16 to each valve leaflet (e.g., the anterior and posterior leaflets). With respect to the state of the heart 1 shown in FIGS. 2A and 2B, the proper coaptation of the valve leaflets 61, which may be due in part to proper position of the papillary muscles 15, may advantageously result in mitral valve operation substantially free of regurgitation/leakage.

As shown in FIG. 2B, the heads of the papillary muscles 15 may be oriented below at least a portion of the mitral valve 6. While FIG. 2B shows positions of two papillary muscles, it should be understood that a ventricle may include any number of papillary muscles. In some cases, the papillary muscles 15 may be positioned directly below or nearly directly below the coaptation line 21 between leaflets and/or portions of leaflets of the mitral valve 6 and/or other valve of the heart. The papillary muscles 151, 15m may be positioned with different orientations with respect to the mitral valve in some cases.

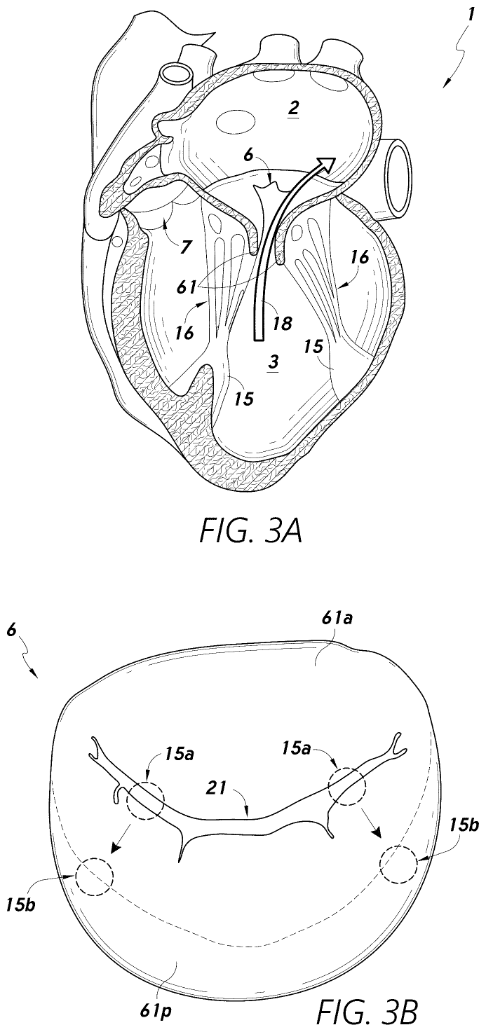

Heart valve disease represents a condition in which one or more of the valves of the heart fails to function properly. Diseased heart valves may be categorized as stenotic, wherein the valve does not open sufficiently to allow adequate forward flow of blood through the valve, and/or incompetent, wherein the valve does not close completely, causing excessive backward flow of blood through the valve when the valve is closed. In certain conditions, valve disease can be severely debilitating and even fatal if left untreated. With regard to incompetent heart valves, over time and/or due to various physiological conditions, the position of papillary muscles may become altered, thereby potentially contributing to valve regurgitation. For example, as shown in FIG. 3A, which illustrates a cross-sectional view of a heart 1′ experiencing mitral regurgitation flow 18, dilation of the left ventricle 3 may cause changes in the position of the papillary muscles 15 that allow flow 18 back from the ventricle 3 to the atrium 2. Dilation of the left ventricle 3 can be caused by any number of conditions, such as focal myocardial infarction, global ischemia of the myocardial tissue, or idiopathic dilated cardiomyopathy, resulting in alterations in the geometric relationship between papillary muscles and other components associated with the valve(s) that can cause valve regurgitation. Functional regurgitation may further be present even where the valve components may be normal pathologically, yet may be unable to function properly due to changes in the surrounding environment. Examples of such changes include geometric alterations of one or more heart chambers and/or decreases in myocardial contractility. In any case, the resultant volume overload that exists as a result of an insufficient valve may increase chamber wall stress, which may eventually result in a dilatory effect that causes papillary muscle alteration resulting in valve dysfunction and degraded cardiac efficiency.

As the heart and/or ventricle dilates, one or more papillary muscles may move away from the coaptation line 21 of the mitral valve and/or a central area of the mitral valve. Dilation of a heart may cause and/or may be characterized by stretching and/or thinning of the walls of the heart. Consequently, an inner volume of one or more chambers of the heart (e.g., the left ventricle 3, as shown in FIG. 3A) may increase.

The positions of the papillary muscles 15 with respect to the mitral valve 6 may affect the functioning of the mitral valve 6. In some cases, as the papillary muscles 15 move away from a central portion of the mitral valve 6, the chordae tendineae 16 tethered between the papillary muscles and the mitral valve 6 may cause the mitral valve 6 to open and/or may prevent the mitral valve 6 from closing. Because the chordae tendineae 16 connect to the papillary muscles 15 (specifically, at tips/heads of the papillary muscles 15), migration of the papillary muscles causes corresponding migration of the chordae tendineae 16, which may cause undesirable force on the leaflets of the mitral valve. FIG. 3B shows the papillary muscles migrating from more-central positions 15a to more-spread-out positions 15b further apart from each other and/or further from a position below the mitral valve 6. In such spread-out positions, the chordae tendineae 16 may pull the leaflets 51 apart such that the leaflets cannot fully coapt. As the papillary muscles move further from the central portion of the mitral valve 6 and/or a coaptation line 21 of the mitral valve 6, the chordae tendineae 16 may cause the leaflets 61 to separate and/or the coaptation line 21 to open. For example, the profile of the mitral valve 6 may be stretched from the natural shape thereof (shown in dashed-line in FIG. 3B) to an expanded, stretched-out shape as shown in FIG. 3B.

Some embodiments disclosed herein provide solutions for treating heart valve disease using minimally invasive procedures and/or without the need for surgical procedures or destroying cardiac tissue. In particular, minimally invasive and/or passive techniques to improve valve performance are disclosed for improving cardiac function. Further, various embodiments disclosed herein provide for the treatment of heart valve disease that can be executed on a beating heart, thereby allowing for the ability to assess the efficacy of the treatment and potentially implement modification thereto without the need for bypass support.

### Papillary Muscle Approximation

The coaptation of mitral valve leaflets can be improved in some cases by repositioning the papillary muscles and the left ventricle to/towards their midline, and therefore procedures implementing such repositioning can be implemented for the treatment of mitral regurgitation. For example, some solutions involve the placement of a band, sling, or other type of tie around the papillary muscles, wherein such band is tightened and/or secured in a manner as to create a constricting barrier around the papillary muscles that serves to approximate the papillary muscles towards one another. Such bands can be encircled about the papillary muscles and/or the trabecular base in such area to form a closed or open loop. The term “band” is used herein according to its broad and ordinary meaning and may refer to any elongate line, tether, tie, sling, ribbon, cord, strip, strand, rope, cable, wire, filament, string, strap, lace, or portion thereof, or other type/form of material used in medical procedures to physically couple anatomy.

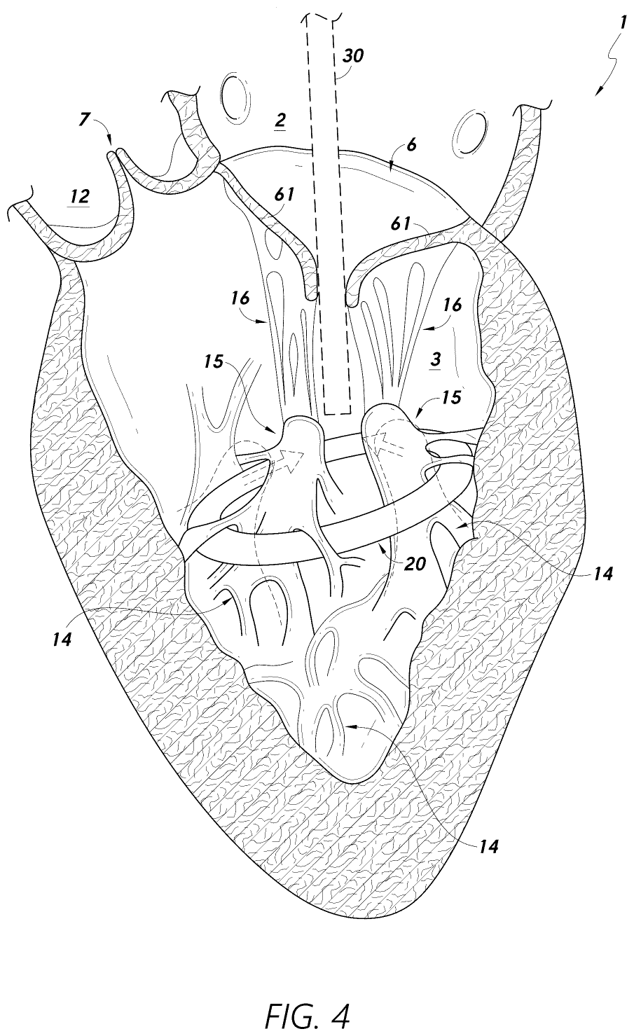

FIG. 4 shows a cutaway view of a left ventricle 3 in which papillary muscles 15 thereof have been approximated using a band device 20 to bring the papillary muscles 15 closer together in a manner as to improve coaptation of the leaflets 61 of the mitral valve 6. Although certain papillary muscle approximation devices, systems, and/or methods/procedures are described herein in the context of left ventricular papillary muscle approximation and/or mitral valve treatments, it should be understood that such examples may be implemented in connection with right ventricular papillary muscle approximation and/or for the purpose of treating tricuspid valve dysfunction. Therefore, any description of the mitral valve herein may be interpreted to refer to the tricuspid valve, and description of the left ventricle and associated papillary muscles can be interpreted to refer to the right ventricle and associated papillary muscles.

Papillary muscle approximation procedures can be implemented in connection with open-heart or minimally-invasive surgery, wherein access to the ventricle may be made via incision in the left atrium, such that access to the ventricle can be made via the mitral valve. For example, FIG. 4 shows an example instrument 30 that may be utilized to perform the approximation of the papillary muscles 15 in the left ventricle 3, such as by implanting/deploying the band device 20 or other papillary-muscle-manipulating device around the papillary muscles 15. Utilization of papillary muscle approximation procedures can result in improved systolic leaflet coaptation, reduction in recurrence of regurgitation, and/or improved left ventricular function.

Various problems may present in connection with certain papillary muscle approximation procedures or solutions. For example, placing a band/sling around the base of papillary muscles within the ventricle, and/or through trabeculae carneae tissue in the area of the base of such papillary muscles, can represent a challenging and/or time-consuming procedure. Furthermore, it may generally be necessary or desirable to execute such procedures with a substantial amount of care when encircling the papillary muscles in order to avoid puncturing the papillary muscles and/or trabeculae, or cause other tissue damage.

### Papillary-Muscle-Approximation Band Navigation/Papillary-Muscle-Circumscribing Instrumentation

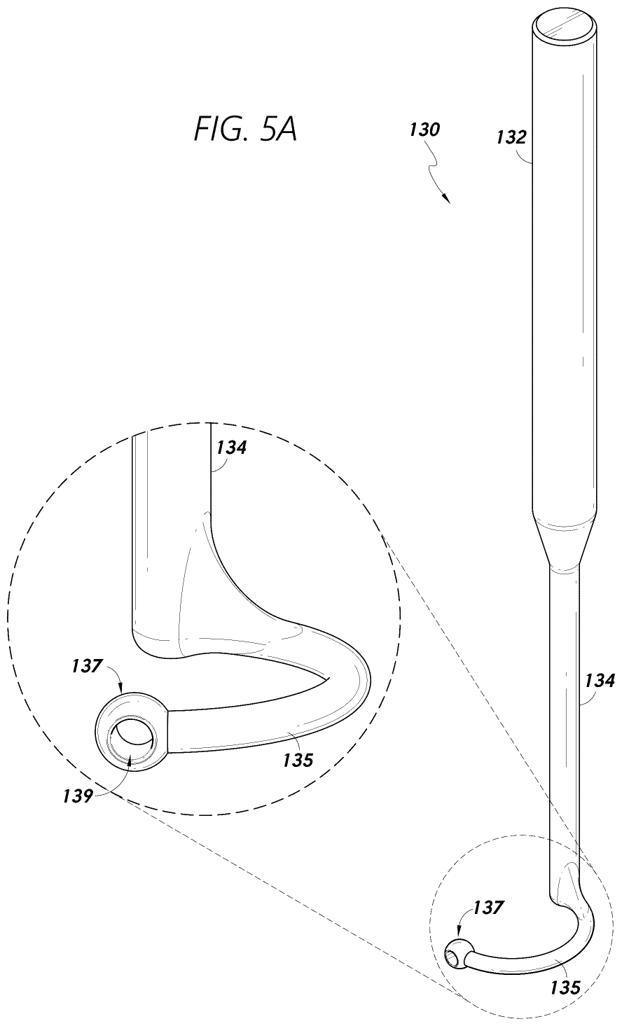

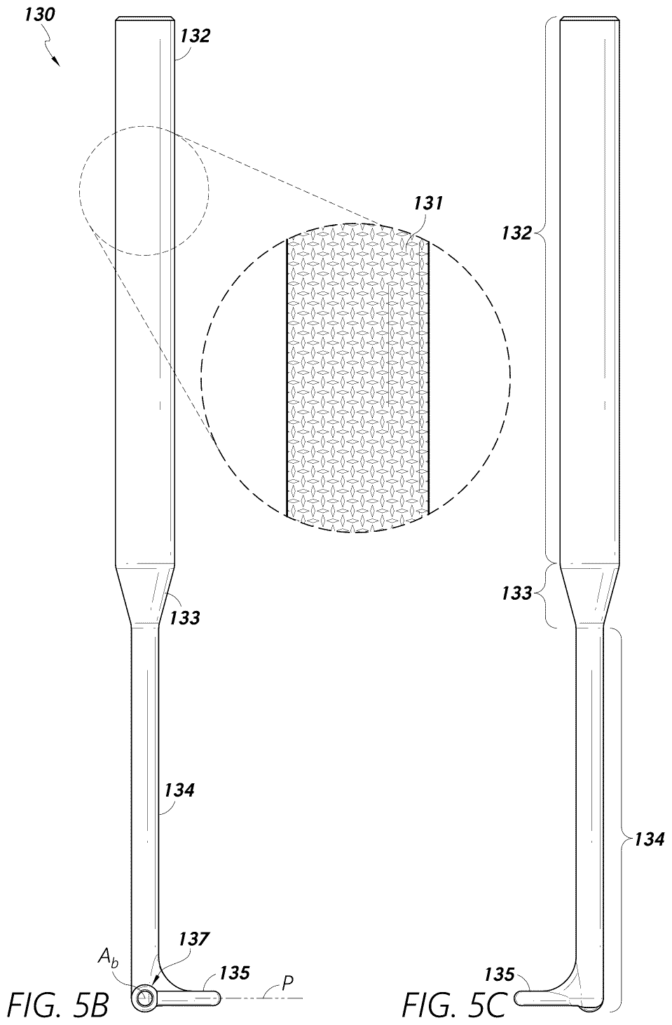

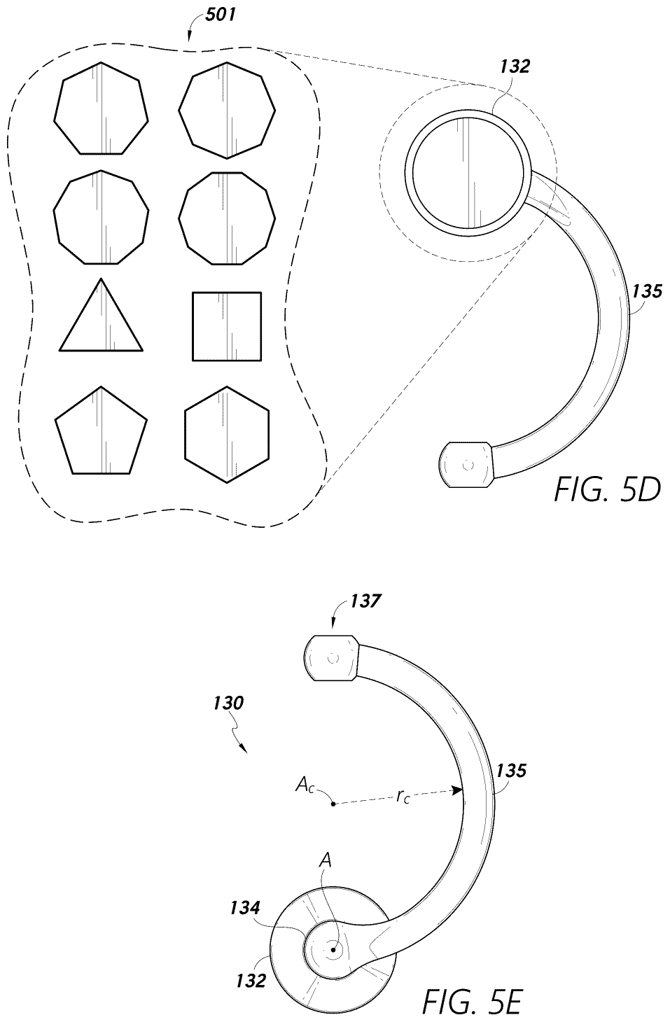

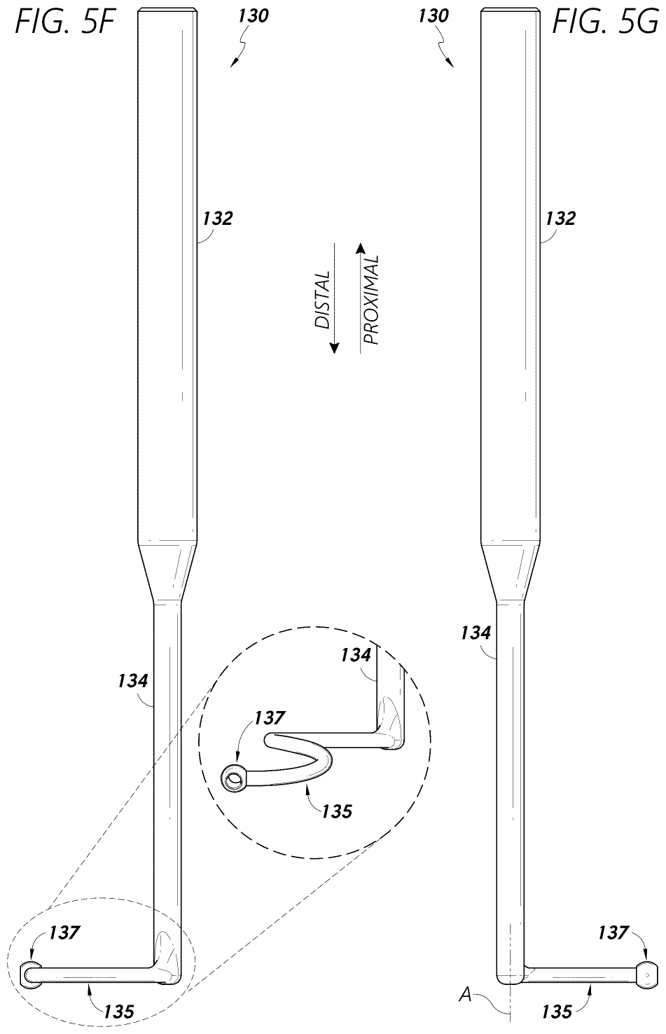

Certain examples disclosed herein relate to instruments that may be implemented/utilized to thread/navigate papillary muscle approximation bands, or similar devices, through/around trabeculae carneae and papillary muscle anatomy within a ventricle. FIGS. 5A-5G provide views of an anatomy-circumscribing instrument 130 in accordance with some examples. The anatomy-circumscribing instrument 130 may comprise a long-shafted threading instrument designed to facilitate navigation of a papillary-muscle-approximating band around a papillary muscle. Use of the instrument 130 may address some of the challenges described above with respect to the threading/navigation of papillary muscle approximating bands around the papillary muscles and/or associated anatomy (e.g., trabeculae carneae). Procedures utilizing the instrument 130 or similar devices can facilitate the threading of a band device through trabeculations of a ventricle or other heart chamber and around papillary muscles to facilitate approximation thereof. The instrument 130 may be composed of any type of material, such as stainless steel.

The instrument 130 includes a distal curved arm 135 that can make the threading/navigation process for a band device relatively more efficient, safe, and/or effective with respect to the navigation of the band through relatively tight areas of the trabecular base of the papillary muscles, for example. The distal arm 135 can further provide increases in the accuracy and speed of threading/navigation of a band device compared to the use of certain other threading tools/instruments, which may suffer from difficulty with respect to the maneuvering of the instrument and/or band through/around the papillary muscles and associated trabeculations that can introduce time-consuming procedural steps. Use of devices/instruments like the instrument 130 can further allow for the placement of a band device relatively close to the ventricular wall while encircling the posterior and anterior papillary muscles, which may help to prevent upward migration of the band and provide added security for the band implant.

The circumscribing tool 130 includes a handle 132, which extends at least a portion of the length of the tool 130. A shaft portion 134 may extend distally from the handle 132 to elongate/extend the device/tool 130 towards the distal end thereof. The curved distal arm 135 emanates from the distal end of the shaft portion 134. For example, as shown in FIG. 5E, the distal arm 135 may project from an axial center A of the device and curve in one direction or another. The curvature of the arm 135 may have any suitable or desirable chirality, which may refer to the handedness/direction of the arm 135. The terms “chirality,” “handedness,” and “direction” with respect to a circumscribing arm of an instrument disclosed herein are used herein according to their broad and ordinary meanings. For example, with respect to a shaft-type instrument including a distal circumscribing/curved arm, the handedness, or chirality, of the arm and/or instrument may be considered right-handed, or clockwise, if with respect to a line of sight along the axis A of the instrument shaft and/or an axis of curvature A_{c} of the curved arm with the proximal end of the instrument/shaft facing the observer and the distal end of instrument/shaft facing away from the observer, following the arm towards a terminating end thereof curves in a clockwise direction, as with the example instrument 130 of FIGS. 5A-5G. If the arm curves in the opposite direction, such arm/instrument may be considered to have left-handed, or counterclockwise, chirality. While the particular examples of FIGS. 5A-5G include an arm 135 with right-handed chirality, it should be understood that circumscriber tools of the present disclosure may have left-handed/counterclockwise chirality and/or may be configured such that the arm 135 can be rotated or otherwise adjusted/articulated to have either left- or right-handed chirality.

The shaft portion 134 that extends distally from the handle 132 may have a step-down diameter relative to the handle 132, which may be desirable to allow the shaft 134 to extend to the base of the papillary muscle while occupying a reduced volume to reduce obstruction of the view of the surgeon and/or otherwise reduce interference with the anatomy of the patient. The tool 130 may include a tapered portion 133 that produces the stepped-down diameter between the handle 132 and the shaft 134. In some implementations, the handle portion 132 and the shaft portion 134 have a common diameter and/or are a continuous and/or integrated form.

The terminating end of the curved arm 135 may have an atraumatic ball/bulb feature 137, which may have an eyelet/channel 139 running therethrough. For example, the arm may terminate in a ball tip 137 that is equipped with an eyelet opening 139 to receive a suture or band. The tip 137 is advantageously atraumatic in that it has a rounded shape, such as a spherical, elliptical, or other shape substantially devoid of edges. As in the illustrated example, the ball 137 may have an at least partially spherical, or spheroid, shape. The eyelet/channel 139 may provide a suture- or band-threading/coupling means, wherein the suture or band may be passed through the channel 139 to couple the suture and/or band thereto. The round shape of the tip 137 can provide an atraumatic tissue contact that reduces the risk of puncturing and/or abrading the papillary muscle tissue and/or adjacent anatomy. Furthermore, the curved spherical surface of the tip 137 can allow for a smooth gliding of the tip through trabeculae muscle strands and/or around the papillary muscle base, which can facilitate the threading process through the relatively tighter areas of ventricular trabeculation. The aperture/channel 139 in the tip 137 may have an axis A_{b} that is normal to the curvature of the arm 135. In some implementations, the axis A_{b} of the channel 139 intersects the axis A of the handle/shaft. The orientation of the channel 139 as normal to the curve of the arm 135 may reduce the risk of the band and/or associated suture(s) becoming decoupled from the tip 137 as the arm 135 passes around and/or through the ventricular anatomy.

The curvature of the distal arm 135 may have any desirable radius r_{c}, and may advantageously be designed to conform to the contour of a target papillary muscle and/or base thereof. The arm 135 may emanate from the axis A of the handle/shaft 132/134 in a plane P that is perpendicular/orthogonal to the axis A of the device/instrument 130. Alternatively, the plane P may be angled with respect to the axis A. The arm 135 may have a circular cross-sectional shape to reduce friction and/or risk of physical interference when the arm is passed through target anatomy. The curvature of the arm 135 may advantageously allow for wrapping of the arm 135 around the target papillary muscle(s).

The instrument 130 is designed to be rotated about the axis A of the handle/shaft to execute a circumscribing path of the tip 137 of the curved distal arm 135 about a papillary muscle base. In some implementations, the torque necessary to rotate the handle 132, in view of the relatively tight space available to the surgeon for manual manipulation due to anatomical constraints and/or other factors, can require a secure grip on the handle 132 by the surgeon. Furthermore, the particular constraints of papillary muscle wrapping procedure can result in the surgeon having only three-finger contact with the handle 132 when rotating the handle 132, and therefore strong grip on the handle 132 may be paramount in some procedures. Therefore, the outer surface 131 of the handle 132 may advantageously be designed to facilitate manual gripping. For example, the outer surface 131 may be textured with topical depressions and/or projections in a manner that facilitates increased purchase between the surgeon's fingers and the handle when gripped.

Furthermore, the cross-sectional shape of the handle may facilitate grip. For example, FIG. 5D shows, in alternate detail image 501, various options of cross-sectional shape of the handle 132 that are alternative implementations compared to the circular shape shown in FIG. 5D. For example, the shapes illustrated may provide relatively flat surface areas that may increase the force area available for force application by the surgeon to the handle. For example, the fewer the number of sides of the cross-sectional shape, the greater the angle of force application between the surgeon's fingers and the handle when applying a rotational force on the handle. Therefore, the triangular, square, and hexagonal shapes may be desirable in some applications. Other example shapes can include pentagonal, heptagonal, octagonal, or nonagonal cross-sectional shapes.

FIGS. 6-1, 6-2, 6-3, 6-4, and 6-5 illustrate a flow diagram for a process 600 for capturing papillary muscle anatomy using a circumscriber instrument in accordance with some examples. FIGS. 7-1, 7-2, 7-3, 7-4, 7-5, 7-6, and 7-7 provide images of the circumscriber instrument 130 and certain anatomy corresponding to operations of the process 600 of FIGS. 6-1, 6-2, 6-3, 6-4, and 6-5 in accordance with some examples.

At block 602, the process 600 involves providing a circumscriber instrument 130, including at least a handle 132 and a curved distal arm 135. The circumscriber instrument 130 may be an implementation of any example circumscriber instrument/tool described herein. The distal arm 135 of the tool 130 may or may not be pre-attached to a band device 120 that is configured to be used to approximate papillary muscles or other anatomy.

As shown in FIG. 7-1, the band 120 may be composed of any type of biocompatible material, such as polytetrafluoroethylene (PTFE) (e.g., Gore-Tex™ expanded PTFE, W.L. Gore). The band 120, in some examples (as shown in FIG. 7), may have a tapered tip 121 design to facilitate ingress into the channel of the tip. In some implementations, the end(s) 121 of the band 120 can be folded or cut to provide a narrowing shape at one or both ends to act as a lead-in feature to facilitate insertion thereof into a channel 139 of the terminating end 137 of the papillary-muscle-circumscribing arm 135 of the tool 130. In some examples, a suture loop 125 is pre-attached to one or both ends 121 of the band 120. The suture loop 125 may be used to couple the band 120 to the tip/channel 137/139 of the curved distal arm 135 of the instrument 130. For example, a suture 129 may be threaded through the eyelet channel 139 of the ball tip 137 of the arm 135, wherein the suture 129 may be coupled to the suture loop 125, such as by looping the suture 129 through the loop 125 and through the channel 139 to thereby physically coupled the band 120 to the tip 137 of the arm 135. A knot may be tied between the suture loop 125 of the band 120 and the suture 129, or the suture loop 125 may be passed through the aperture/channel 139 of the arm terminator 137 such that the band 120 is passed through the loop 125 on an opposite side of the channel 139 to secure the band 120 and suture loop 125 to the arm terminator 137. That is, the suture loop 125 may be inserted through the channel 139, wherein the band 120 may be passed through the portion of the loop 125 that is passed through and out of the channel 139 to thereby secure the suture loop 125 and band 120 to the tip 137. Additionally or alternatively, the band 120 and/or suture loop 125 may be passed through the channel 139 and tied in a knot to prevent the band 120 and/or suture loop 125 from being pulled back through the channel/aperture 139.

In some implementations, the band 120 itself is passed through and/or into the channel 139 of the arm tip 137. The band 120 may have an at least partially flattened form (e.g., not entirely cylindrical/circular in cross-sectional shape), which may advantageously resist pulling through and out of the channel 139 when the band 120 is inserted therein. Where the band is inserted through the aperture/channel 139, the end 121 thereof may be pulled a distance of 1 to 2 inches or more past the channel outlet to prevent the band 120 from being pulled back out through the channel 139.

At block 604, the process 600 involves advancing the curved distal arm 135 of the circumscribed tool 130 to the base of a papillary muscle 15a, as shown in FIG. 7-2. For example, the curved arm 135 of the threader/circumscribed tool 130 may be placed adjacent to the base of the papillary muscle 15a on a side of the instrument associated with the chirality of the curved arm, such that the curved arm 135 may be advanced around the base of the papillary muscle 15a from such position. When placed at the base of the papillary muscle 15a, the ball tip 137 of the instrument 130 may or may not be pre-attached to a band device (not shown in FIG. 7-2 for visual clarity). FIG. 7-2 shows the instrument 130 placed with the curved distal arm 135 in the area of the base of a papillary muscle 15a.

At block 606, the process 600 involves maneuvering the tip 137 of the distal arm 135 around the papillary muscle 15a with the band 120 coupled to the tip 137 to thereby pass the band 120 around the papillary muscle. For example, the curved arm 135 may be dimensioned to wrap around a single papillary muscle, but not both, such that a procedure to wrap the band 120 around both papillary muscles may require first passing the band around the first papillary muscle 15a and subsequently passing the same and/or opposite end 121 of the band 120 around the other papillary muscle 15b after repositioning and/or reconfiguring the instrument 130. When advancing the arm 135 and/or band 120 around the papillary muscle 15a, the tip 137 of the arm 135 may be threaded through trabeculae 14 in the area of the base of the papillary muscle 15a, which may provide a good hold on the base of the papillary muscle 15a without tearing the tissue.

The advancement of the band 120 around the papillary muscle 15a in connection with block 606 may be performed either by pre-attaching the band 120 to the tip 137 and advancing the band 120 around the papillary muscle 15a along with the advancement of the tip 137 forward around the papillary muscle, or alternatively, the tip 137 may be advanced toward around the papillary muscle 15a without the band 120 pre-attached thereto, wherein after the tip 137 has passed around the papillary muscle 15a, the band 120 may be coupled to the tip 137, such that subsequent retraction/rotation of the arm 135 and/or tip 137 around the papillary muscle 15a in the backward direction can pull the band 120 around the papillary muscle.

FIG. 7-3 shows a pre-attached band 120 being advanced around the papillary muscle 15a in the forward direction with the tip 137 of the instrument 130. As shown, the band 120 can be pre-attached to the tip 137 prior to wrapping the arm 135 around the papillary muscle 15a, such that when the tip 137 of the arm 135 is advanced forward around the papillary muscle 15a, it brings the end 121 of the band 120 along through the path/threading of the arm 135 to thereby pass the band 120 around the papillary muscle 15a and/or secure the band 120 in anatomy (e.g., trabeculae) through which the arm 135 is threaded.

FIG. 7-4 shows the alternative implementation in which the tip/arm 137/135 is passed around the papillary muscle 15a prior to attachment of the band 120 to the tip 137. After advancement of the tip 137 around the papillary muscle 15a, wherein such advancement may be through trabeculae carneae features 14, the end portion 121 of the band 120 may be attached to the tip 137 of the arm 135 in some manner. For example, the band 120 may be snared/attached via a suture loop 125 associated with an end portion 121 thereof to the aperture/channel 139 through the tip 137. For example, in some implementations, once the curved/ball distal tip 137 of the arm 135 encircles the papillary muscle 15a, such that the tip 137 is exposed at the base of the papillary muscle 15a, a suture 129 may be threaded through the opening/channel 139 in the tip 137, wherein the suture 129 may be used to couple the band 120 to the tip 137, either directly, or via looping/coupling with/through a suture loop 125 that is pre-attached to the end portion 121 of the band 120. For example, the suture 129 may be tied to the suture loop 125 to secure the suture loop 125, and therefore the band 120, to the tip 137. Once the band 120 has been coupled to the tip 137, the instrument may be rotated back around the papillary muscle 15a in the direction opposite of the initial advancement around the papillary muscle, to thereby pull the band 120 at least partially back through the path initially traversed by the tip 137 around the papillary muscle 15a. The result may be that the curved arm 135 is brought back from around the papillary muscle 15a, such that the band 120 is presently passed around the papillary muscle 15a with an end thereof in the area of the base of the papillary muscle where the instrument 130 was initially positioned (see FIG. 7-2). As the instrument 130 is rotated back, pulling the suture/band 120 through the threaded anatomy as it encircles the papillary muscle 15a, the opposite end of the band may be held upward to prevent bunching of the band 120 during such step(s).

With reference to both FIG. 7-3 and FIG. 7-4, once the band 120 end portion 121 has been passed around the papillary muscle 15a, such end portion 121 may be pulled further to further draw the band 120 through the path around the papillary muscle. In some implementations, the end 121 of the band 120 may be brought upward through the mitral valve to an area where the band can be manually manipulated. That is, the band 120 may be sufficiently long to allow for excess length from both ends thereof to be disposed out of the ventricle, with a medial portion of the band passing around the papillary muscle 15a, to allow for manual manipulation of the band ends.

At block 608, the process 600 involves detaching the instrument 130 from the band 120 and moving the instrument away from the first papillary muscle 15a to the base of a second papillary muscle 15b. For example, the suture(s) coupling the band 120 to the tip 137 may be cut or otherwise released/untied in some manner to detach the instrument 130 from the band 120. In some implementations, the band 120 may be unknotted and/or otherwise decoupled from the tip 137 by pulling the band 120 through the aperture/channel 139 of the atraumatic tip 137. When the band 120 is decoupled from the tip 137, the end 121 of the band 120 may be pulled sufficiently far from the base of the papillary muscle 15a to reduce the risk that the band 120 may be pulled back around/through the base of the papillary muscle 15a, thereby undoing the previous step(s). For example, the end 121 of the band may be pulled all the way out of the patient's body in some implementations.

At block 610, the process 600 involves maneuvering the curved distal arm 135 of the instrument 130 around the second papillary muscle 15b to pass the band 120 around the second papillary muscle 15b. For example, as described above, such advancement of the band around papillary muscles may be implemented by pre-attaching the band 120 to the tip 137 of the instrument 130 and advancing the tip 137 and band 120 around the second papillary muscle 15b, or by first passing the tip 137 around the second papillary muscle 15b, after which the tip 137 is snared and coupled to the band 120, such that retracting/rotating the arm 135 back around the second papillary muscle 15b pulls the band 120 around the second papillary muscle 15b. In both example implementations, the end 121 of the band 120 that is attached to the tip 137 and passed around the papillary muscle 15b may be either the first end that was initially passed around the first papillary muscle 15a, or may be the opposite end of the band 120. In some implementations, the instrument 130 comprises a hinge or other feature that allows for the curved distal arm 135 to be swung or otherwise articulated in a manner as to change the chirality/handedness of the arm 135 to accommodate wrapping the arm around the second papillary muscle 15b in the opposite direction of the rotation around the first papillary muscle 15a.

FIGS. 7-5 shows the implementation in which the band 120 is pre-attached to the tip 137 of the instrument 130 prior to advancement around the second papillary muscle 15b. For example, the instrument 130 may be withdrawn from the patient and the band 120 may be re-attached to the tip 137 of the instrument 130 prior to re-insertion of the instrument 130 into the ventricle and to the base of the second papillary muscle 15b. In some implementations, the instrument 130 is swapped out for a second instrument that has a curved distal arm that is curved in the opposite direction of the first instrument, which may facilitate passing the opposite end of the band 120 from the end that passed around the first papillary muscle 15a around the second papillary muscle 15b.

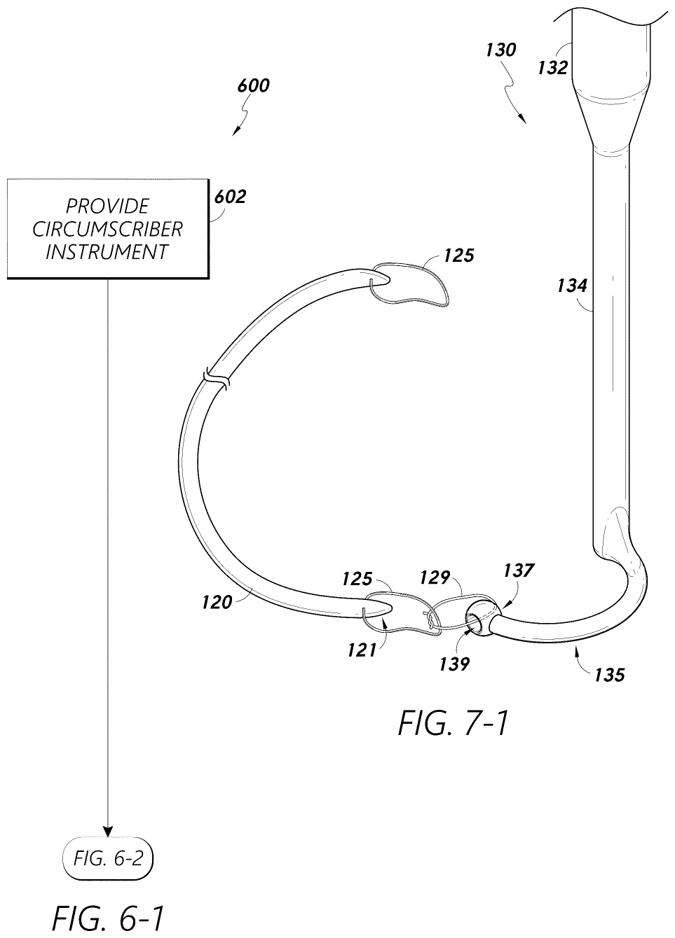

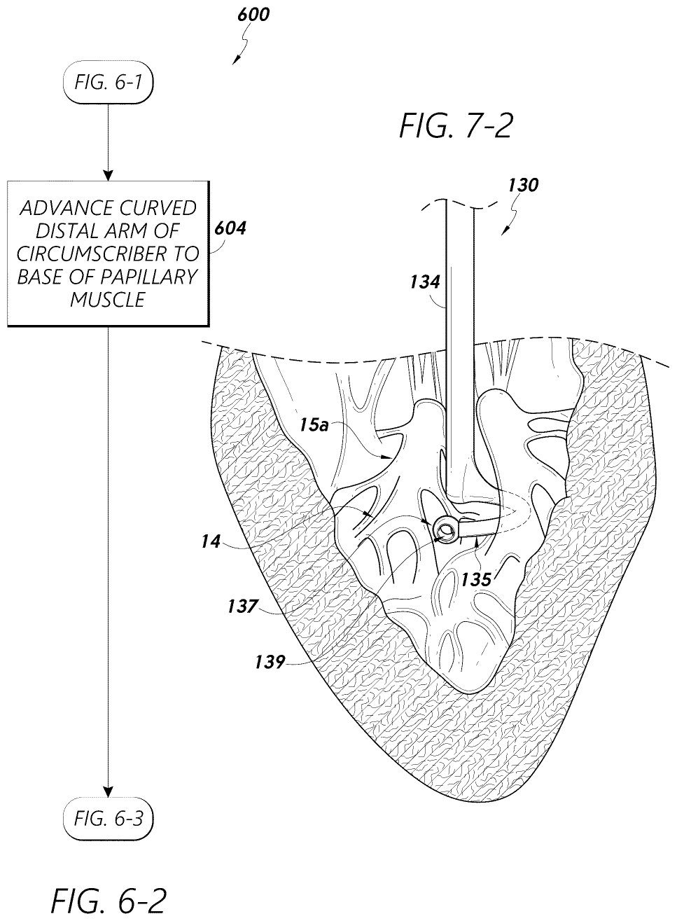

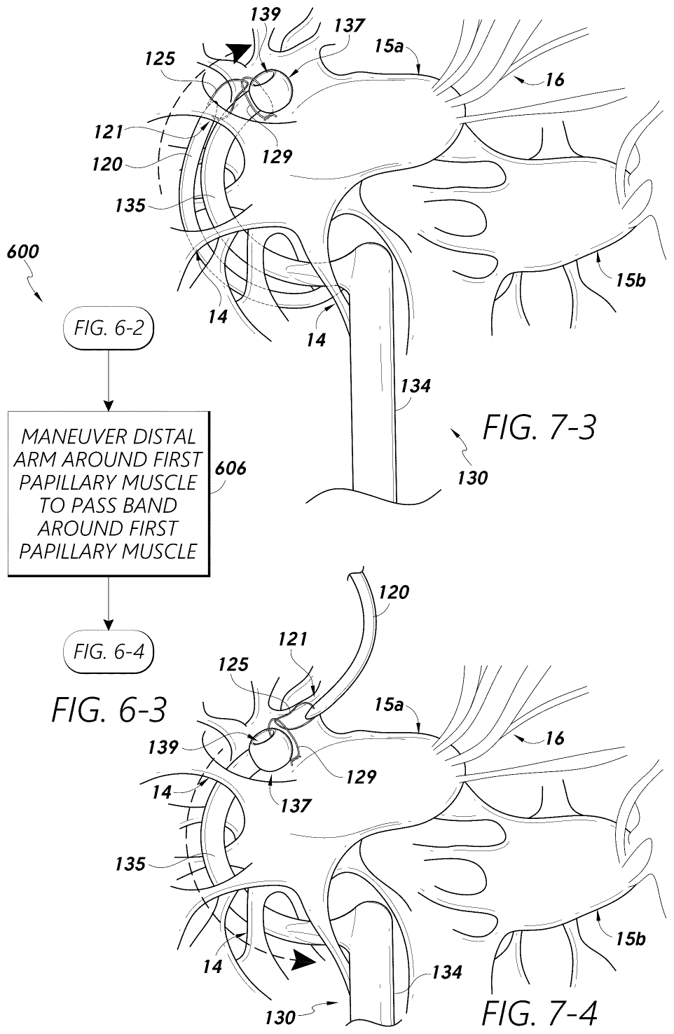

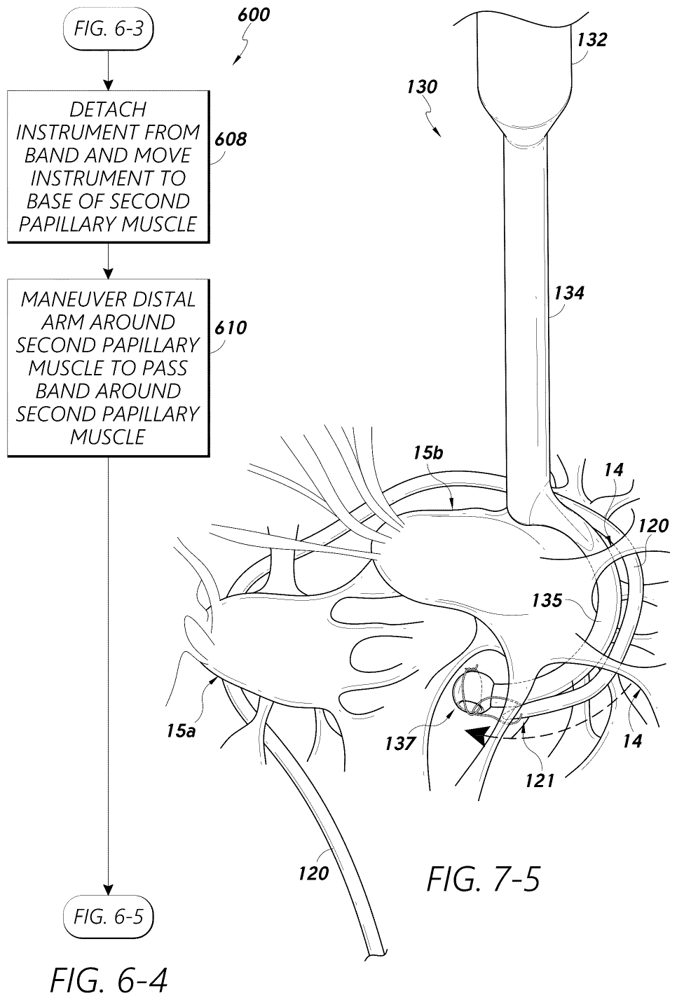

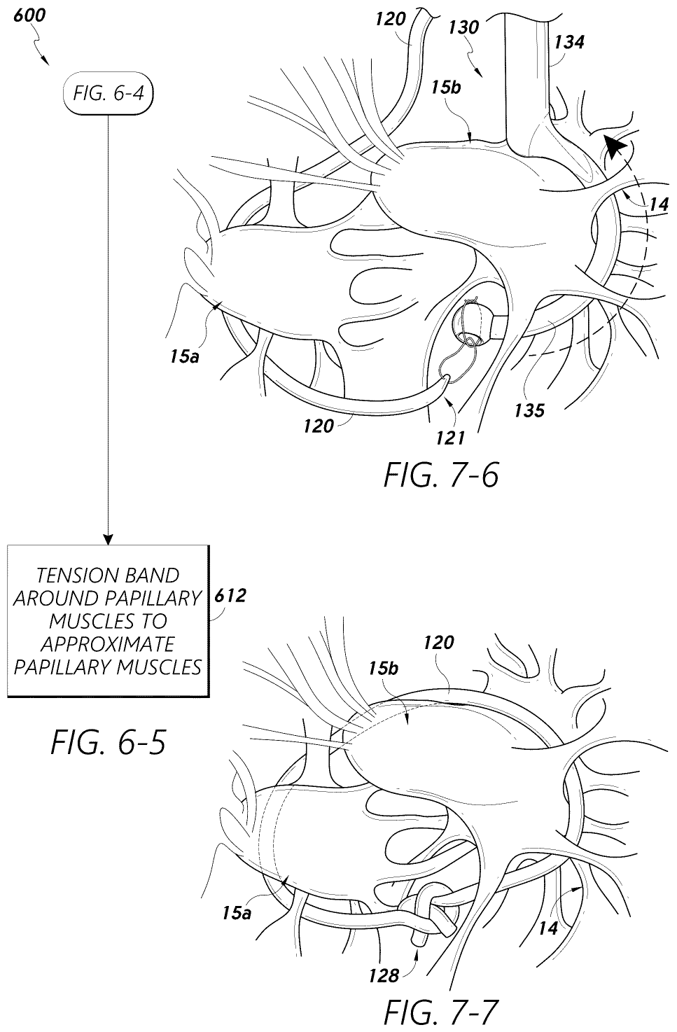

FIG. 7-6 shows the implementation in which the curved arm 135 is passed around the second papillary muscle 15b without the band 120 being attached to the tip 137 thereof, wherein after the tip 137 passes around the second papillary muscle 15b, the tip 137 is snared and/or otherwise coupled to the band 120 to allow for the tip 137 to be rotated back in the opposite direction to thereby pull the band 120 through the path traversed by the curved arm 135 around the second papillary muscle 15b.

At block 612, the process 600 involves tensioning the band 120 around the captured papillary muscles 15a, 15b to thereby approximate the papillary muscles towards one another to some degree. For example, both ends of the band 120 may be pulled/tensioned to a desired degree and coupled in the tensioned state to thereby retain the papillary muscles in the constrained/approximated position. Once the tension is locked in the band 120, the end tails of the band 120 may be trimmed/cut to reduce the length thereof.

FIG. 7-7 shows the band 120 tightened around the first 15a and second 15b papillary muscles and fixed in a tensioned configuration to maintain the constrained/approximated positioning of the papillary muscles. The tension in the band may be fixed using any type of tension locking means/mechanism 128, such as a clip, knot, clamp, clasp, buckle, or the like. The band 120 encircles the trabecular base of the first 15a papillary muscle and the second 15b papillary muscle, creating a complete sling that brings both papillary muscles into relatively close proximity and/or contact with one another.

Papillary muscles circumscriber tools disclosed herein may have any suitable or desirable shape or configuration. For example, handle portions of circumscriber tools disclosed herein can assume a variety of design configurations and sizes to facilitate the threading/wrapping process in a relatively efficient and effective manner to achieve the desired valve correction.

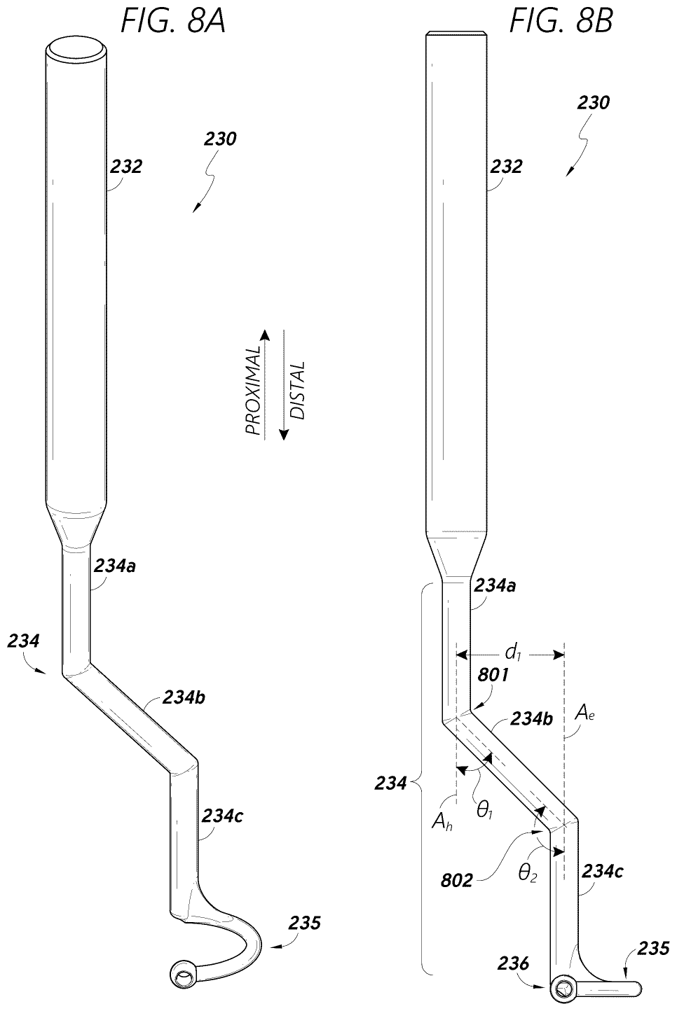

FIGS. 8A and 8B show perspective and side views, respectively, of an anatomy-circumscribing instrument 230 that may be implemented in connection with any of the examples disclosed herein. The instrument 230 includes a handle portion 232 and a shaft portion 234 that extends distally from the handle portion 232 to a distal end, which has associated therewith a curved arm 235, as described in detail above. The shaft portion 234 of the instrument 230, unlike the straight-shaft examples shown in FIGS. 5A-5G, is bent or deflected in some manner away from the axis Aₕ of the handle 232, with respect to at least a portion of the shaft length.

The shaft portion 234 may include one or more bends 801 that deflect the shaft 234 away from the axis A, wherein the bends/angles (0) help to avoid obstructing the physician's view when executing a papillary muscle wrapping procedure as described herein. For example, with the axis Aₕ of the handle 232 being removed/offset from the axis/orientation A, of the distal end of the instrument, the surgeon's hands may advantageously be placed out of the area of view of the base of the target papillary muscle(s), improving the ability of the surgeon to manipulate the handle 232 to navigate the distal arm 235 around the papillary muscles.

The particular example shown in FIGS. 8A and 8B includes a shaft portion 234 that has two bends 801, 802 at complementary angles θ₁, θ₂, such that the shaft portion 234 has a first straight portion 234a that is aligned with the axis Aₕ of the handle 232, a bent portion 234b that deflects away from the axis Aₕ of the handle 232, and a second straight portion 234c that is substantially in parallel with the axis Aₕ of the handle 232 but offset therefrom my some distance d₁. However, it should be understood that any number/configuration of bends and/or angles thereof may be implemented in the shaft portion 234 to achieve the desired relative positioning of the handle 232 and distal end 236. For example, the number and/or angle of bends may be selected/determined based at least in part on the size of the opening in the chest through which access is made to the ventricle, or the particular anatomy of the patient. In some implementations, the shaft portion 234 may be malleable at least to some degree to allow the practitioner to manually bend the shaft portion 234 to achieve the desired shape thereof.

In some implementations, papillary-muscle-circumscriber tools/instruments in accordance with aspects of the present disclosure may include certain articulation/hinge features, which may allow for the handle of an instrument to be articulated with respect to a distal curved arm of the instrument.

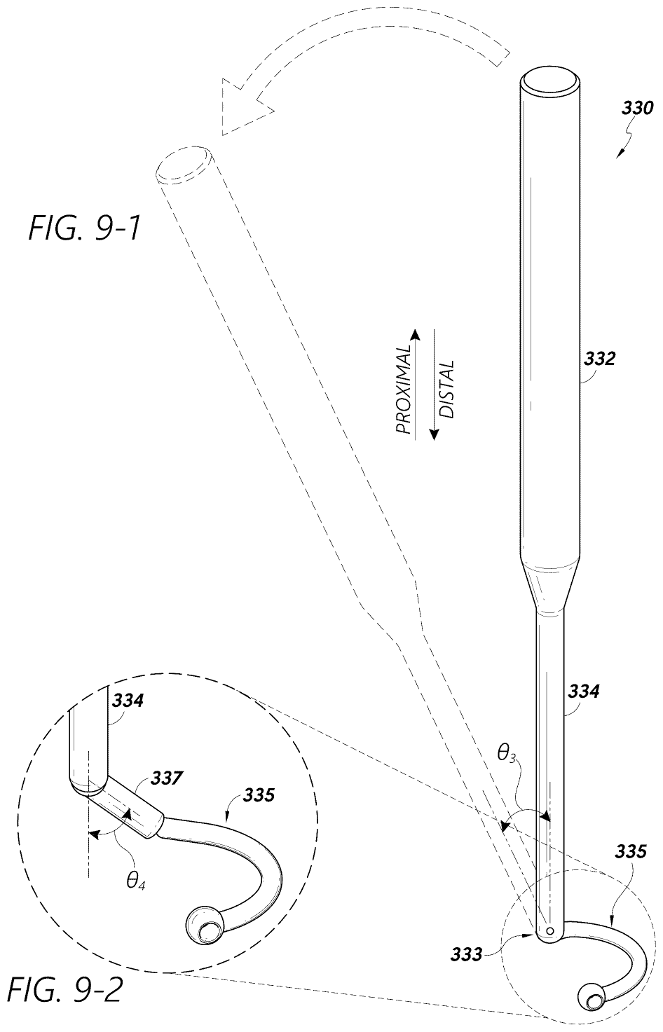

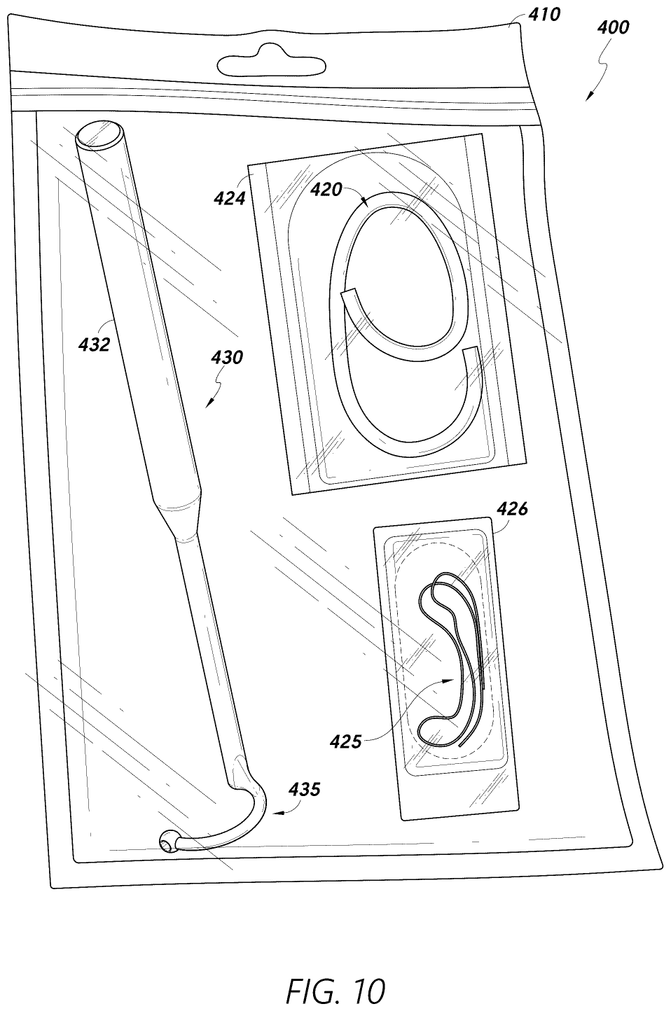

FIGS. 9-1 and 9-2 show anatomy-circumscribing instruments having distal deflection/articulating features in accordance with some examples. The instrument 330 of FIG. 9-1 includes a handle 332 and/or shaft 334, wherein a distal end of the instrument is associated with a curved arm 335, which may have the features of any similar components described herein. The instrument 330 further includes an articulation feature 333, such as a hinge or other mechanism that allows for the handle/shaft 332/334 to rotate/articulate relative to the curved arm 335, as shown. Such articulation may provide flexibility to the surgeon with respect to moving the handle 332 relative to the visual field of the surgeon to thereby increase the visibility of the distal curved arm 335 and/or anatomy in the area thereof. In FIG. 9-1, the articulation feature 333 comprises a singular rotating hinge. However, it should be understood that articulation features of the present disclosure may include any number of hinge or other articulation features. The hinge feature 333 may allow for the angle θ₃ between the shaft 334 and the base of the curved arm 335 to be selectably adjusted.

FIG. 9-2 shows an alternative design that allows for the distal curved arm 335 to be displaced/offset and/or articulated with respect to the distal end of the instrument. As shown in FIG. 9-2, the curved arm 335 is coupled to the distal end of the shaft via a deflected extension arm 337, which may serve to project the base of the curved arm 335 away from the axis of the shaft portion of the instrument, which may assist in improving visibility and/or otherwise positioning the curved arm in a desired position relative to the shaft portion to facilitate wrapping around papillary muscle. The extension arm 337 may be angled by any suitable or desirable angle θ₄ relative to the shaft 334. The bend areas associated with the extension 337 on either end thereof may or may not be articulatable. That is, each bend may have associated therewith a hinge or other articulation feature to allow for rotation of the components relative to one another in such areas. In some implementations, the bends are not rotatable/articulatable, but rather provide a rigid positioning of the curved arm 335 relative to the shaft 334.

FIG. 10 shows a papillary-muscle-approximation kit 400 in accordance with some examples. For example, any circumscriber tool described herein may be produced and/or manufactured as part of the kit 400 for use in certain surgical procedures. In some implementations, the kit 400 may comprise a pouch or other container 410, which may include any flexible and/or rigid components. Although a pouch 410 is shown in FIG. 10, it should be understood that the kit 400 may comprise a tray and/or other container.

The kit 400 includes a circumscriber tool 430, including a handle 432 and a curved distal arm 435. The circumscriber tool 430 may be similar to any of the example circumscriber tools described herein. The kit 400 may further include a band 420, which may comprise ePTFE (e.g., Gore-Tex™ MePTFE, W.L. Gore) tubing or similar material or structure, as with any other example band implant device disclosed herein. The band may be contained within a separate pouch or container 424 within the container/pouch 410, or may be loose within the container/pouch 410. The band 420 may or may not have pre-attached suture loop(s) associated with one or more ends thereof, as described herein. The kit 400 may further comprise one or more sutures 425, which may be contained in a separate pouch/container 426 within the pouch/container 410, or may be loose within the pouch/container 410. Although the kit 400 is shown with the circumscriber tool 430, the band 420, and the suture 425, it should be understood that the kit 400 may include additional components, and/or one or more of the illustrated components may be omitted. For example, the kit 400 may comprise the instrument 430 and the band 420, but not the suture 425, or the instrument 430 and the suture 425, but not the band 420.

### Papillary Muscle Spacing

Various solutions are presented herein for approximating papillary muscles to improve patient outcomes. However, certain anatomy of the ventricular chamber may present certain challenges with respect to papillary muscle approximation. Ventricular anatomy can make the securing of papillary muscles and/or using means or mechanisms for approximating or bringing the property muscles together relatively challenging. For example, papillary muscles shape, size, and/or the number of heads that are associated with the papillary muscles may vary from patient-to-patient. In some cases, partial approximation of the papillary muscles, as an alternative to bringing the papillary muscles fully together into flush contact with one another can relieve excess tethering on the mitral valve leaflets and significantly restore leaflet mobility without requiring full approximation of the papillary muscles. While fully approximating the papillary muscles into physical contact with one another can provide a relatively simple solution in some instances, optimized tensioning on the leaflets can be associated with a relative position of the papillary muscles that is not fully approximated, but rather maintains some desired distance between the papillary muscles. Examples of the present disclosure provide solutions that allow for a known distance between papillary muscles to be set when performing papillary muscle approximation.

In accordance with some papillary muscle approximation solutions, the papillary muscles are approximated together without knowledge of the distance between the papillary muscles once approximated or whether it is necessary to fully approximate the papillary muscles to improve coaptation of the valve leaflets. That is, some solutions involve simply implementing band tying and cinching/tensioning steps without regard to particular positioning of the papillary muscles at a more optimal distance apart.

In some implementations, aspects of the present disclosure provide papillary muscle approximation solutions that set the papillary muscles at a known relative position to one another, wherein the papillary muscles are spaced a certain distance to provide improved outcomes/results. Such solutions may be referred to herein as partial papillary muscle approximation which, compared to full papillary muscle approximation, wherein the papillary muscles are brought into physical contact with one another, can provide a safe, measurable, and/or concise solution for setting an optimal distance between papillary muscles in connection with the surgical procedure. Partial papillary muscle approximation using known spacing, as described in detail below, can provide the surgeon relatively greater control and flexibility to reduce leaflet tension, and restore and preserve the balance between the papillary muscles, chordae tendineae, and/or leaflets. Such solutions can thereby improve mitral valve leaflet coaptation. Papillary muscle approximation, or partial approximation, in connection with aspects of the present disclosure, can be implemented in conjunction with an annuloplasty ring, wherein such combined intervention can improve outcomes in terms of reducing or eliminating mitral regurgitation, while also inducing positive left ventricular remodeling in some cases.

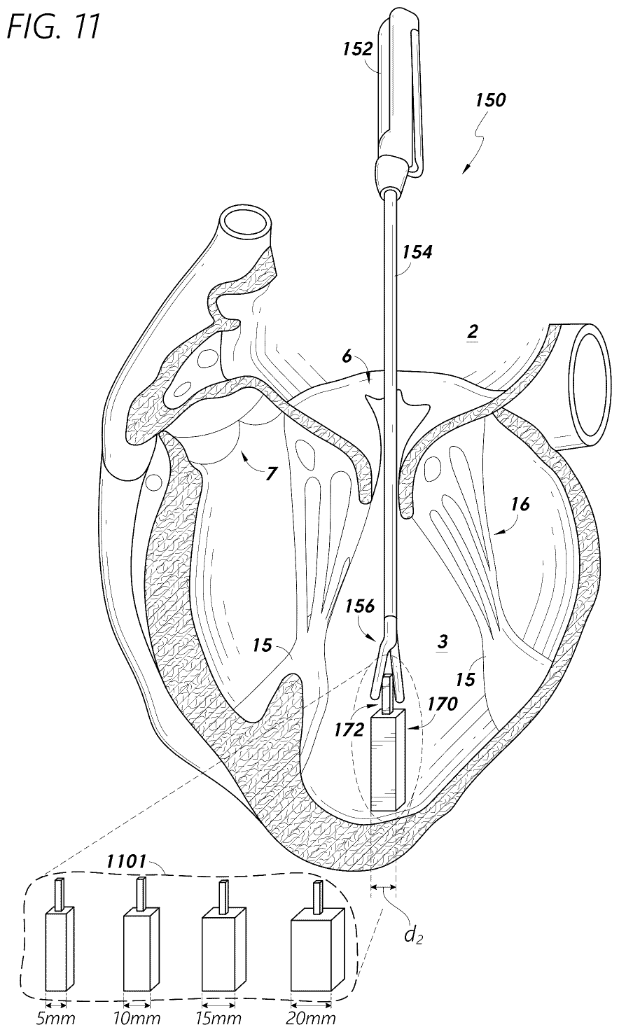

Examples of the present disclosure provide spacer devices having known dimensions that can be placed between papillary muscles during an approximation procedure to provide control over the approximation distance. FIG. 11 shows a cutaway view of a heart ventricle 3 having a spacer device 170 placed between papillary muscles 15 thereof in accordance with some examples. Use of a spacer device, such as the spacer 170 shown in FIG. 11, can help establish a normal/desirable annulus-to-papillary-muscle alignment. The spacer 170 can help supplement a papillary muscle approximation process in a manner as to make the process more efficient, safe, and/or effective, and may enable the practitioner to have more control over the repair and optimization thereof. For example, a spacer as shown in FIG. 11 may be used in connection with the process 600 disclosed above.

FIG. 11 shows the spacer device 170 being placed between papillary muscles 15 in the ventricle 3 of the heart 1. For example, the spacer 170 may be placed and/or held by an elongated instrument 150, which may include a distal grasper or other securing means 156 for securing the spacer 170 to the instrument 150 for placement. In some embodiments, the spacer 170 is integrated with the shaft 154 of the instrument 150. The spacer 170 may be placed between the papillary muscles 15 and held in place during at least a portion of a papillary muscle approximation procedure, such as the procedure 600 described above, to thereby prevent the papillary muscles from encroaching on the volume occupied by the spacer form 170. The spacer 170 may therefore force a separation distance between the papillary muscles 15 as the muscles are approximated, such as by the tightening of a band wrapped around the papillary muscles 15 as described in detail herein.

The use of the spacer 170 and/or instrument 150 as a means of setting a known distance between the papillary muscles 15 can provide the surgeon a mechanism to enable fine-tuned adjustment to optimally improve the efficacy of a papillary muscle approximation procedure. The ability to control the papillary muscles distance can allow for a greater number or percentage of potential patients to be eligible for valve treatment through papillary muscle approximation.

The instrument 150 may be used to insert the spacer/sizer 170 in between the papillary muscles 15 during, for example, an approximation procedure as described herein. The spacer 170 may comprise a rigid body with a known dimension d₂, wherein the space d₂ is designed to set a fixed distance between the papillary muscles. The spacer 170 provides a stopper/stop that gives the physician tension feedback when tensioning the papillary muscle approximation band at a known distance/value between the papillary muscles.

The spacer 170 can come in any suitable or desirable width, shape, and/or volume. For example, various sizes are shown in the example details 1101 in FIG. 11, including a spacer having an about 5-mm width, wherein the width dimension is the dimension between the papillary muscles when the spacer is placed. Example 10-mm, 15-mm, and 20-mm sizes are also shown. It should be understood that spacers having sizes less than about 5 mm or greater than about 20 mm may be implemented as well, as may spacers having any value between the discrete values shown in detail 1101 in FIG. 11. Various methods or mechanisms may be implemented to establish the desired distance between the papillary muscles 15, wherein the spacer 170 is designed to present such spacing/distance between the papillary muscles. For example, the surgeon may measure the aortic annulus and divide such value by some parameter to determine the desired papillary muscle distance. For example, the spacing may be determined to be approximately 80% of the distance of the aortic annulus dimension/diameter. In some implementations, the distance between the trigones may be measured, and such distance (or a calculated distance proportional thereto) may be used as the papillary muscle separation distance provided by the spacer form 170.

The instrument 150 may comprise a grasping instrument, or other instrument that can be used to deliver and/or retrieve the spacer 170. In some implementations, the spacer is used with a dedicated delivery system which may comprise a rigid shaft having a proximal end that serves as a handle 152 and a distal end/tip 156 that interfaces with the spacer 170 in some manner, such as by grasping, or otherwise coupling/holding to the spacer 170. In some implementations, the spacer 170 and shaft 154 of the instrument 150 can be a single integrated form/part.

The spacer 170 can comprise silicone, plastic, metal alloy, or other material shaped into a three-dimensional volume. The volume/form of the spacer 170 may advantageously have relatively smooth surfaces, and may advantageously be devoid of sharp edges to reduce the risk of tissue damage associated with the utilization of the spacer 170 and/or instrument 150 as the device is being manipulated/moved between the papillary muscles 15 or elsewhere within the ventricle or other cardiac anatomy.

When approximating the papillary muscles 15 to the spacer 170, the resulting repair of the cardiac anatomy may be monitored to determine whether the spacer 170 is the correct size. For example, a water test, syringe bulb fill test, or other test known to those having skill in the art may be implemented to see if the valve leaflets 61 collapse in the manner desired when the spacer 170 is present and the papillary muscles 15 are drawn against the spacer. If the valve leaflets do not coapt properly, the spacer 170 may be removed and replaced with a spacer having different size or dimension(s), wherein further testing may be implemented until the appropriate/desired papillary muscles spacing is achieved.

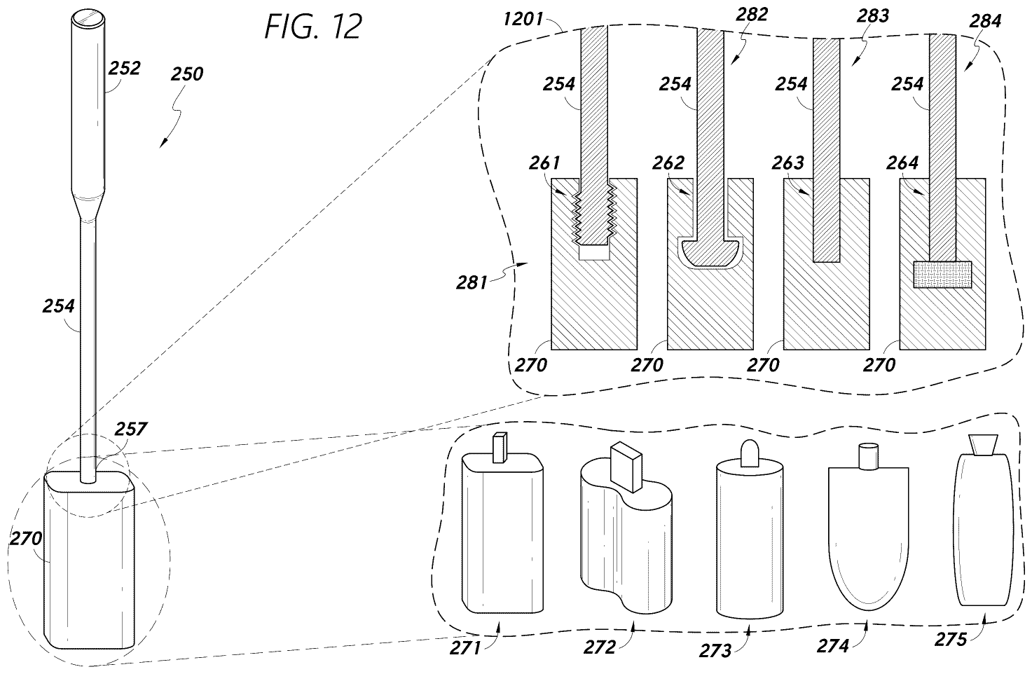

FIG. 12 shows spacer instruments in accordance with some examples. FIG. 12 shows a spacer instrument 250 having a handle 252 and a spacer form 270. The instrument 250 may further comprise a shaft 254 extending distally from the handle 252 to the proximal base of the spacer 270, wherein the shaft 254 may be coupled and/or integrated with the spacer 270 in any suitable or desirable manner. For example, FIG. 12 shows various alternative implementations of the coupling between the shaft 254 and the spacer 270. Such implementations are presented as examples only, and it should be understood that the shaft 254 may be coupled to the spacer 270 in any suitable or desirable manner. For example, the shaft 254 may be a single unitary form integrated with the spacer form 270 (e.g., molded from a common material).

In one implementation, FIG. 12 shows a threaded coupling 261 between the shaft 254 and the spacer 270, wherein the shaft 254 may comprise male threads configured to mate with and be secured within a recess comprising corresponding female threads associated with the spacer form 270. Alternatively, the shaft 254 may comprise female threads, whereas the spacer 270 may comprise male threads. Furthermore, although the example 281 in image detail 1201 shows the shaft 254 projecting into a recess of the spacer 270 and being threaded therewith, in some implementations, the spacer form 270 may comprise a threaded male projection configured to be received within a female threaded receptacle of the shaft 254.

In another implementation 282, the image detail 1201 of FIG. 12 shows a mechanical interference coupling 262 between the shaft 254 and the spacer 270. For example, the spacer 270 may include a recess configured to receive a distal portion of the shaft 254, wherein the recess includes certain undercut features configured to present an interference lock with phalange feature(s) of the shaft 254 to prevent proximal withdrawal of the shaft 254 from the spacer 270 or other separation of such components.

In another implementation 283, the image detail 1201 of FIG. 12 shows a mechanical frictional fit coupling 263 between the shaft 254 and the spacer 270. For example, the spacer 270 may include a recess dimensioned to present a press-fit coupling interface with the distal portion of the shaft 254, wherein the shaft 254 may be forced into the recess, such that a frictional fit between the shaft 254 and the recess constrains withdrawal of the shaft 254 from the spacer 270 or other decoupling. The recess may comprise tapered walls, for example, which may provide increased resistance to penetration of the shaft 254 as the shaft 254 is advanced further into the recess, such interference presenting frictional force between the shaft 254 and the spacer recess to secure such components to one another.

In another implementation 284, the image detail 1201 of FIG. 12 shows a magnetic coupling 264 between the shaft 254 and the spacer, wherein magnetic components may be associated with one or both of the distal end of the shaft 254 and/or the spacer 270 or recess receptacle portion thereof, such that magnetic force holds the shaft 254 to the spacer 270 when the shaft 254 is introduced into the receptacle or other mating structure of the spacer 270. Although the examples in image 1201 of FIG. 12 shows the shaft 254 extending into a receptacle or other feature of the spacer 270, it should be understood that any of such examples may be implemented without the shaft 254 extending into the spacer 270, or with the spacer 270 potentially extending into a receptacle or structural feature of the shaft 254.

FIG. 12 shows example shapes that the spacer form 270 may take. As referenced above, a spacer in accordance with aspects of the present disclosure may have any suitable or desirable shape with respect to axial or diametrical cross-section, or any volumetric features or forms suitable for providing a spacer volume. Although any shape or size may be implemented, certain examples are illustrated for reference, including a rectangular cylinder 271, a peanut or figure-eight shaped cylinder 272, a circular cylinder 273, a top-shaped form 274, and/or an axially convex cylinder 275.

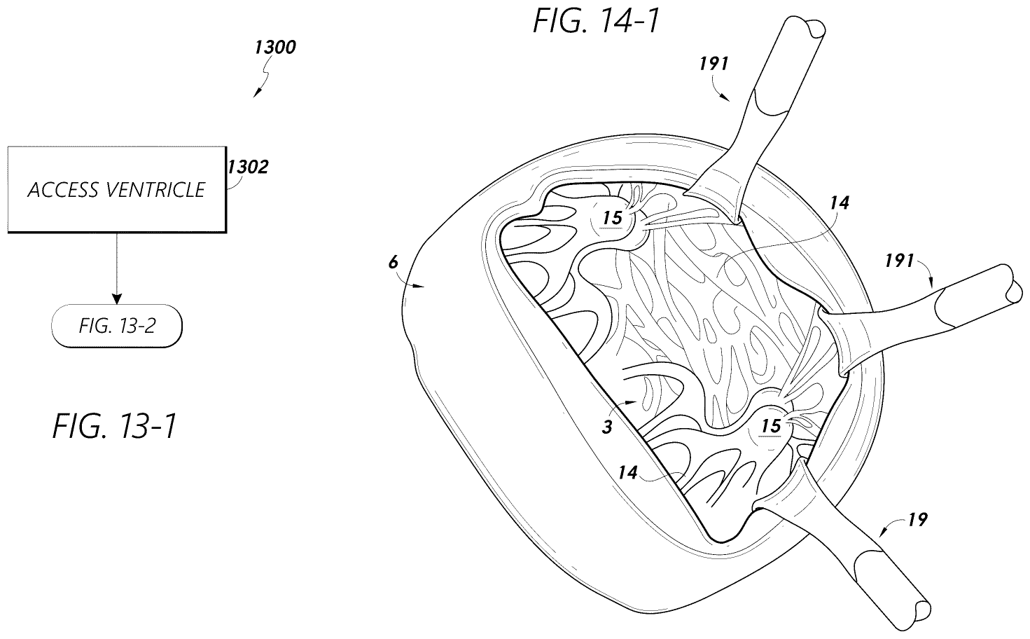

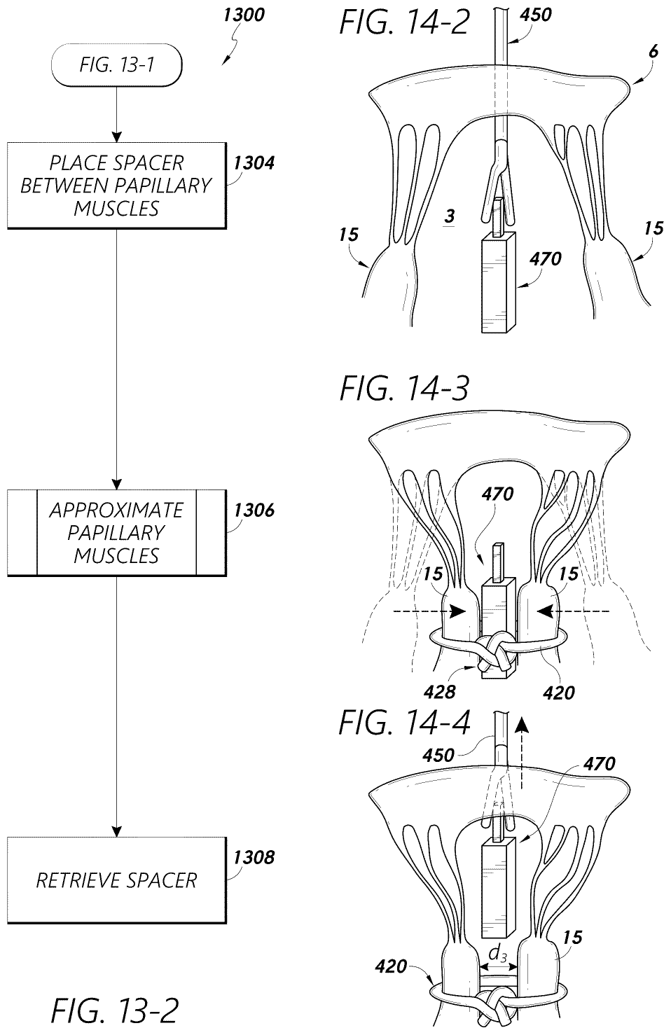

FIGS. 13-1 and 13-2 illustrate a flow diagram for a process 1300 for approximating papillary muscle anatomy 15 using one or more spacers 470 in accordance with some examples. FIGS. 14-1, 14-2, 14-3, and 14-4 provide images of instrumentation and certain anatomy corresponding to operations of the process 1300 of FIGS. 13-1 and 13-2 in accordance with some examples.

At block 1302, the process 1300 involves accessing a target ventricle of a patient's heart. For example, such access may be made using a surgical incision through the chest of the patient. An example implementation is shown in FIG. 14-1, which shows access to a ventricle 3 (e.g., left atrium) through an atrioventricular heart valve 6 (e.g., mitral valve). The process 1300 may involve opening the chest of the patient and cutting into the atrium (e.g., left atrium) to reveal the access atrioventricular valve (e.g., mitral valve). The valve leaflets 61 may be pulled back using retractor instrumentation 191 to reveal the target ventricle 3 and provide an open channel thereto. That is, the left atrium may be incised, and the mitral valve 6 exposed by use of one or more atrial retractors, where the retractor(s) may be inserted between the posterior and anterior leaflets to expose the papillary muscles 15 below.

At block 1304, the process 1300 involves placing a spacer 470 between first and second papillary muscles 15 within the left ventricle 3 (or right ventricle where access is made through the tricuspid valve for tricuspid valve repair). FIG. 14-2 shows the spacer 470 introduced into the ventricle 3 between the anterolateral and posteromedial papillary muscles. The particular spacer used may be selected based on the desired gap size for the particular patient anatomy.

At block 1306, the process 1300 involves approximating the papillary muscles using an appropriate papillary muscle approximation procedure as described in detail herein. For example, such procedure may involve wrapping a band device 420 around the papillary muscles 15 and tensioning the band device to a desired tension to pull the papillary muscles closer to one another. FIG. 14-3 shows the band 420 wrapped around the papillary muscles 15 and held at a tensioned configuration with the papillary muscles 15 brought together into contact with, and separated by, the spacer 470.

Any papillary muscle approximation procedure described herein or known to those having ordinary skill in the art may be implemented in connection with the subprocess 1306 and/or process 1300. Papillary muscle approximation in connection with block 1306 may involve using a papillary muscle circumscriber instrument, which may comprise a handle and a curved distal arm configured to be threaded through anatomy of the ventricle around a papillary muscles, wherein the device may be used to navigate through the chordae tendineae, trabeculae carneae, and/or other anatomy within the ventricle to encircle the papillary muscles individually or collectively. Once the curved arm of the circumscriber instrument has been rotated to encircled a papillary muscle, rotation of the circumscriber may be reversed to withdraw the curved arm from around the papillary muscle, wherein the band may be navigated around the papillary muscle through attachment to the curved arm (e.g., distal tip thereof) either while the arm is being advanced around papillary muscle or while the arm is being retracted/reversed back around the papillary muscle. Such papillary muscle wrapping may be implemented sequentially for both papillary muscles 15.

When the band 420 is wrapped around the papillary muscles 15, the band 420 may be tensioned to draw the papillary muscles 15 together, wherein such approximation of the papillary muscles 15 may be limited by the presence of the spacer volume 470 between the papillary muscles 15, which stops the papillary muscles 15 from further movement/approximation once the papillary muscles 15 are in contact with the spacer form 470. Both ends of the band 420 may be tensioned and pulled taught to approximate the papillary muscles 15 to the spacer 470. The band 420 can be temporarily tied with a suture or other device/mechanism, at which point the valve 6 may be tested, such as by doing a field test with a bulb syringe, or other suitable test, to determine whether the leaflets 61 coapt in a desirable manner. If the results of the test are suitable, the band 420 may be secured in place at the present tension. If not, the spacer 470 may be replaced with another spacer having different size/dimensions.

At block 1308, the process 1300 involves retrieving the spacer 470. For example, with the papillary muscles 15 sufficiently-tightly approximated and spaced by the distance d₃ set by the spacer form 470, the band 420 may be fixed in the tensioned configuration and the spacer 470 may be removed from the ventricle, thereby leaving the remodeled papillary muscles 15 and/or ventricle in place. FIG. 14-4 shows the papillary muscles 15 approximated closer together with the spacer 470 having been removed. The band 420 may be retained in-place around the papillary muscles 15 to improve valve function as described herein. It may be desirable to remove the spacer 470 to avoid the presence of a foreign body in the ventricle. Although the process 1300 is described as involving removing the spacer 470, in some implementations, a spacer form may be maintained in-place in the ventricle to maintain the proper spacing distance between the papillary muscles postoperatively. In such cases, the process 1300 may involve anchoring the spacer form to the cardiac anatomy, such as to the papillary muscle(s). Removing the spacer may be preferred to avoid interfering with the function of the ventricle.

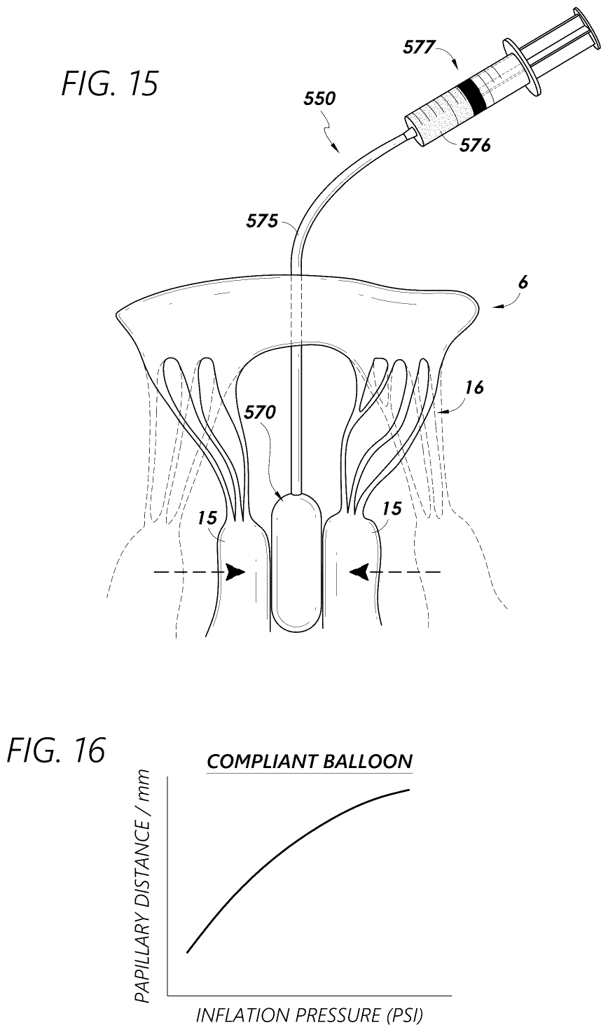

Although some examples are disclosed herein in which a papillary muscles spacer device has a fixed volume or dimensions(s), it should be understood that examples of the present disclosure may be implemented with spacer devices having variable and/or controllable volumes. FIG. 15 shows a balloon spacer instrument 550 in accordance with some examples, wherein the balloon spacer 550 provides a variable spacer volume 570 for use during a surgical procedure, such that the surgeon may dynamically control the volume of the spacer to a desired volume/dimension to produce a desired outcome.

The instrument 550 shown in FIG. 15 may comprise a balloon spacer component or portion 570, which may have a fluid channel 575 associated therewith that is fluidly-coupled to a reservoir 577 that is controllable to modify the amount of the fluid 576 that fills/enters the balloon 570 to thereby determine the volume of the balloon 570. The balloon 570 may be a compliant or non-compliant balloon. For example, a compliant balloon may be implemented in which the material of the balloon is inelastic and therefore the volume of the balloon has a set maximum beyond which the balloon will not further expand. Such implementations of a non-compliant balloon may be desirable in instances where a target papillary muscle spacing volume or distance is known, such that when the balloon is filled, the volume of the balloon occupies a known space and distance/dimension. Therefore, the surgeon may simply fill the balloon to achieve the known spacing. The reservoir 577 may have a syringe-type configuration, or any other type of pump configuration, which may be controllable either manually or robotically/electro-mechanically.

Alternatively, a compliant balloon may be implemented in which the outer membrane of the balloon stretches in a manner that is proportional to the pressure level within the balloon. Unlike the non-compliant balloon implementations, which provide a relatively narrow window of expansion for the balloon portion, compliant implementations can allow for a wider range of spacing, wherein the surgeon may introduce an amount of fluid into the balloon associated with a desired spacing. Such spacing may therefore be dynamically adjusted through fluid irrigation and aspiration during the procedure to achieve the desired results, which may be determined using any valve leaflet coaptation testing mechanism known to those having skill in the art.

FIG. 16 shows a graph demonstrating distance/diameter of a compliant balloon spacer device relative to balloon inflation pressure in accordance with some examples. For example, the relationship between the inflation pressure of the balloon and the dimensions of the balloon may be known and referenced to determine the desired inflation pressure for the balloon to achieve the desired papillary muscle distance. As represented in the graph of FIG. 16, the relationship between balloon volume, which is a surrogate for papillary muscle distance in the relevant surgical procedures, and balloon inflation pressure may not be exactly linear, but may require increasing degrees of pressure increase to effect volume increase in the balloon as inflation pressures increase.

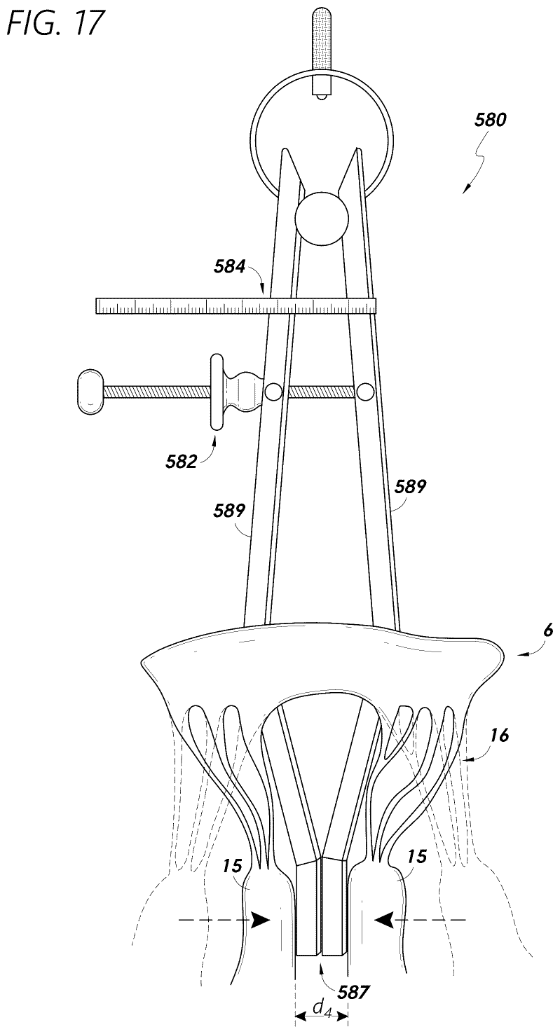

FIG. 17 shows a caliper papillary muscle spacer device 580 in accordance with some examples. The instrument of FIG. 17 provides an alternative mechanism for allowing a surgeon to set a known distance between papillary muscles 15 when performing a papillary muscle approximation procedure. The instrument of FIG. 17 may be considered a caliper spacer in that it comprises a rack and pinion mechanism 582, or other mechanical actuator, which may be actuated to expand the known spacing of the distal portion 587 of the instrument 580, which is disposed/placed as a spacer component between the papillary muscles 15. The instrument 580 may pass through the mitral valve 6 (or tricuspid valve) and/or out of the atrium, depending on the surgical implementation. The caliper instrument 580 may allow for the spacing of the spacer portion 587, which is determined by the position of the actuator 582, to be locked in place at a known spacing d₄ during a papillary muscle approximation procedure. In some implementations, the instrument 580 includes a visual feature/marker 584 that indicates the spacer distance d₄ based on the actuator 582 configuration/position.

### ADDITIONAL DESCRIPTION OF EXAMPLES

Depending on the example, certain acts, events, or functions of any of the processes or algorithms described herein can be performed in a different sequence, may be added, merged, or left out altogether. Thus, in certain examples, not all described acts or events are necessary for the practice of the processes.

Provided below is a list of examples, each of which may include aspects of any of the other examples disclosed herein. Furthermore, aspects of any example described above may be implemented in any of the numbered examples provided below.

Example 1: A surgical device comprising a handle, a shaft extending distally from the handle, and a curved arm projecting from a distal end of the shaft.

Example 2: The surgical device of any example herein, in particular example 1, wherein the curved arm projects in a plane that is orthogonal to an axis of at least one of the handle or the shaft.

Example 3: The surgical device of any example herein, in particular example 1 or example 2, wherein the curved arm is curved with respect to an axis of at least one of the handle or the shaft.

Example 4: The surgical device of any example herein, in particular any of examples 1-3, wherein the curved end comprises an atraumatic terminal tip.

Example 5: The surgical device of any example herein, in particular example 4, wherein the terminal tip is at least partially spherical in shape.

Example 6: The surgical device of any example herein, in particular example 4 or example 5, wherein the terminal tip has an aperture therethrough.

Example 7: The surgical device of any example herein, in particular example 6, wherein the aperture is oriented normal to a curvature of the curved arm.

Example 8: The surgical device of any example herein, in particular example 6 or example 7, wherein an axis of the aperture intersects an axis of at least one of the handle or the shaft.

Example 9: The surgical device of any example herein, in particular any of examples 1-8, wherein a distal tip of the curved arm is sutured to a band device.

Example 10: The surgical device of any example herein, in particular example 9, wherein the distal tip of the curved arm is sutured to the band device via a first suture that passes through an aperture in the distal tip of the curved arm, the first suture being coupled to a second suture that is threaded through an end portion of the band device.

Example 11: The surgical device of any example herein, in particular example 10, wherein the second suture is configured in a suture loop.

Example 12: The surgical device of any example herein, in particular any of examples 1-11, wherein the curved arm includes a straight portion that extends from the distal end of the shaft, and the straight portion sets a curved portion of the curved arm off from the distal end of the shaft.

Example 13: The surgical device of any example herein, in particular any of examples 1-12, wherein the shaft has one or more bends.

Example 14: The surgical device of any example herein, in particular example 13, wherein the one or more bends comprises a first bend between a first straight portion of the shaft that is coaxial with the handle and an angled portion of the shaft that is angled relative to an axis of the handle, and a second bend between the angled portion of the shaft and a second straight portion of the shaft that has an axis that is parallel with the axis of the handle.

Example 15: The surgical device of any example herein, in particular any of examples 1-14, further comprising an articulation feature configured to facilitate articulation of the curved arm relative to the distal end of the shaft.

Example 16: The surgical device of any example herein, in particular example 15, wherein the curved arm includes an angled projection that extends from the distal end of the shaft and positions a curved portion of the curved arm at a radially and axially offset position relative to an axis and position of the distal end of the shaft.

Example 17: A method of any example herein, in particular approximating papillary muscles, the method comprising advancing a shaft of a surgical device into a left ventricle through a mitral valve, the surgical device comprising a curved arm associated with a distal end of the shaft, and rotating a handle of the surgical device in a first direction to cause a tip of the curved arm to pass at least partially around a first papillary muscle within the left ventricle, thereby passing a first portion of a band that is coupled to the curved arm at least partially around the first papillary muscle.

Example 18: The method of any example herein, in particular example 17, wherein said causing the tip of the curved arm to pass at least partially around the first papillary muscle involves threading the tip of the curved arm behind one or more trabeculae carneae columns of the left ventricle.

Example 19: The method of any example herein, in particular example 17 or example 18, further comprising prior to said advancing the shaft into the left ventricle, coupling the band to the curved arm, wherein the first direction is associated with a direction of curvature of the curved arm.

Example 20: The method of any example herein, in particular example 19, wherein said coupling the band to the curved arm comprises passing an end of the band through an aperture in the tip of the curved arm.

Example 21: The method of any example herein, in particular example 19 or example 20, wherein said coupling the band to the curved arm comprises coupling an end of the band to the tip of the curved arm via one or more sutures.

Example 22: The method of any example herein, in particular example 21, wherein the one or more sutures comprises a first suture formed in a loop and sutured to the end of the band, and a second suture that couples the first suture to the tip of the curved arm.

Example 23: The method of any example herein, in particular any of examples 17-22, further comprising: when the band is not coupled to the curved arm, rotating the handle a second direction opposite the first direction, thereby advancing the tip of the curved arm at least partially around the papillary muscle, and after said rotating the handle in the second direction, coupling the band to the curved arm, wherein said rotating the handle in the first direction causes the curved arm to pull the first portion of the band back around at least a portion of the first papillary muscle.

Example 24: The method of any example herein, in particular example 23, wherein said coupling the band to the curved arm comprises passing an end of the band through an aperture in the tip of the curved arm.

Example 25: The method of any example herein, in particular example 23 or example 34, wherein said coupling the band to the curved arm comprises coupling an end of the band to the tip of the curved arm via one or more sutures.

Example 26: The method of any example herein, in particular example 25, wherein the one or more sutures comprises: a first suture formed in a loop and sutured to the end of the band, and a second suture that couples the first suture to the tip of the curved arm.

Example 27: The method of any example herein, in particular any of examples 17-26, further comprising, after said passing the first portion of the band at least partially around the first papillary muscle, decoupling the first portion of the band from the curved arm.

Example 28: The method of any example herein, in particular example 27, further comprising, after said decoupling the first portion of the band from the curved arm, pulling the first portion of the band out of the left ventricle.

Example 29: The method of any example herein, in particular example 27 or example 28, further comprising, after said decoupling the first portion of the band from the curved arm: coupling a second portion of the band to the curved arm, and causing the tip of the curved arm to pass at least partially around a second papillary muscle within the left ventricle, thereby passing the second portion of the band at least partially around the second papillary muscle.

Example 30: The method of any example herein, in particular example 29, wherein the first portion of the band and the second portion of the band are the same.

Example 31: The method of any example herein, in particular example 29 or example 30, wherein the first portion of the band is associated with a first end of the band and the second portion of the band is associated with an opposite end of the band.

Example 32: The method of any example herein, in particular any of examples 17-31, further comprising tensioning the band to cause the first papillary muscle to be approximated to a second papillary muscle.

Example 33: A surgical device comprising a handle, a shaft extending distally from the handle, and a spacer form associated with a distal end of the shaft, the spacer form dimensioned to be placed between first and second papillary muscles to set an approximation limit between the first and second papillary muscles.

Example 34: The surgical device of any example herein, in particular example 33, wherein the spacer form comprises a rigid body having a width dimension between about 4-25 mm.

Example 35: The surgical device of any example herein, in particular example 33 or example 34, wherein the shaft is detachably coupled to the spacer form by attachment means.

Example 36: The surgical device of any example herein, in particular example 35, wherein the attachment means comprises at least one of machined threading, a pressure-fitting receptacle, or a magnet.

Example 37: The surgical device of any example herein, in particular example 35 or example 36, wherein the spacer form has a rectangular prism shape.

Example 38: The surgical device of any example herein, in particular any of examples 35-37, wherein the spacer form has a cylindrical shape.

Example 39: The surgical device of any example herein, in particular any of examples 33-38, wherein the spacer form comprises a balloon fluidly coupled to a fluid reservoir.

Example 40: The surgical device of any example herein, in particular example 39, wherein the balloon is compliant.

Example 41: The surgical device of any example herein, in particular example 39 or example 40, wherein the balloon is non-compliant.

Example 42: A method of approximating papillary muscles, the method comprising advancing a spacer form into a left ventricle through a mitral valve, placing the spacer form between first and second papillary muscles of the left ventricle, and bringing the papillary muscles together against the spacer form.

Example 43: The method of any example herein, in particular example 42, further comprising securing the first and second papillary muscles against the spacer form.

Example 44: The method of any example herein, in particular example 43, further comprising, after said securing the first and second papillary muscles against the spacer form, removing the spacer form from the left ventricle.

Example 45: The method of any example herein, in particular any of examples 42-45, further comprising, after said brining the papillary muscles together against the spacer form, determining whether first and second leaflets of the mitral valve properly coapt.

Example 46: The method of any example herein, in particular example 45, further comprising, when it is determined that the first and second leaflets of the mitral valve do not properly coapt: removing the spacer form, placing another spacer form between the first and second papillary muscles, and brining the papillary muscles together against the other spacer form.

Example 47: The method of any example herein, in particular example 45 or example 46, further comprising, when it is determined that the first and second leaflets of the mitral valve properly coapt: securing the first and second papillary muscles against the spacer form, and removing the spacer form from the left ventricle.

Example 48: The method of any example herein, in particular any of examples 45-47, wherein said determining whether the first and second leaflets properly coapt comprises performing a fill test in the left ventricle.

Example 49: The method of any example herein, in particular any of examples 33-48, further comprising injecting fluid into the spacer form to cause the spacer form to inflate to an expanded volume.

Example 50: The method of any example herein, in particular example 49, further comprising, after said injecting fluid into the spacer form, determining whether first and second leaflets of the mitral valve properly coapt.

Example 51: The method of any example herein, in particular example 50, further comprising, when it is determined that the first and second leaflets of the mitral valve do not properly coapt, adjusting an amount of fluid in spacer form.

Example 52: The method of any example herein, in particular example 50 or example 51, further comprising, when it is determined that the first and second leaflets of the mitral valve properly coapt: securing the first and second papillary muscles against the spacer form, and removing the spacer form from the left ventricle.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A surgical device comprising:
a handle;
a shaft extending distally from the handle; and
a curved arm projecting from a distal end of the shaft.

2. The surgical device of claim 1, wherein the curved arm projects in a plane that is orthogonal to an axis of at least one of the handle or the shaft.

3. The surgical device of claim 1, wherein the curved arm is curved with respect to an axis of at least one of the handle or the shaft.

4. The surgical device of claim 1, wherein the curved arm comprises an atraumatic terminal tip.

5. The surgical device of claim 4, wherein the terminal tip is at least partially spherical in shape.

6. The surgical device of claim 4, wherein the terminal tip has an aperture therethrough.

7. The surgical device of claim 6, wherein the aperture is oriented normal to a curvature of the curved arm.

8. The surgical device of claim 6, wherein an axis of the aperture intersects an axis of at least one of the handle or the shaft.

9. The surgical device of claim 1, wherein a distal tip of the curved arm is sutured to a band device.

10. The surgical device of claim 9, wherein the distal tip of the curved arm is sutured to the band device via a first suture that passes through an aperture in the distal tip of the curved arm, the first suture being coupled to a second suture that is threaded through an end portion of the band device.

11. The surgical device of claim 10, wherein the second suture is configured in a suture loop.

12. The surgical device of claim 1, wherein:
the curved arm includes a straight portion that extends from the distal end of the shaft; and
the straight portion sets a curved portion of the curved arm off from the distal end of the shaft.

13. The surgical device of claim 1, wherein the shaft has one or more bends.

14. The surgical device of claim 13, wherein the one or more bends comprises:
a first bend between a first straight portion of the shaft that is coaxial with the handle and an angled portion of the shaft that is angled relative to an axis of the handle; and
a second bend between the angled portion of the shaft and a second straight portion of the shaft that has an axis that is parallel with the axis of the handle.

15. The surgical device of claim 1, further comprising an articulation feature configured to facilitate articulation of the curved arm relative to the distal end of the shaft.

16. The surgical device of claim 15, wherein the curved arm includes an angled projection that extends from the distal end of the shaft and positions a curved portion of the curved arm at a radially and axially offset position relative to an axis and position of the distal end of the shaft.

17. A surgical device comprising:
a handle;
a shaft extending distally from the handle; and
a spacer form associated with a distal end of the shaft, the spacer form dimensioned to be placed between first and second papillary muscles to set an approximation limit between the first and second papillary muscles.

18. The surgical device of claim 17, wherein the spacer form comprises a rigid body having a width dimension between about 4-25 mm.

19. The surgical device of claim 17, wherein the shaft is detachably coupled to the spacer form by attachment means.

20. The surgical device of claim 19, wherein the attachment means comprises at least one of machined threading, a pressure-fitting receptacle, or a magnet.
